(19) United States
(12) Patent Application Publication
(10) Pub. No.: US 2024/0000797 A1
(43) Pub. Date: Jan. 4, 2024

(54) BIODEGRADABLE POLYMER DELIVERY SYSTEM FOR EXTENDED DELIVERY OF TESTOSTERONE

(71) Applicant: TOLMAR INTERNATIONAL LIMITED, Dublin 2 (IE)

(72) Inventors: Amy VAN HOVE, Fort Collins, CO (US); Dileep JANAGAM, Fort Collins, CO (US); Gregory FIELDSON, Fort Collins, CO (US)

(21) Appl. No.: 18/029,443

(22) PCT Filed: Sep. 24, 2021
(86) PCT No.: PCT/IB2021/058743

§ 371 Date: Mar. 30, 2023

Related U.S. Application Data
(60) Provisional application No. 63/085,868, filed on Sep. 30, 2020.

(51) Int. Cl. A61K 31/568; A61K 47/34; A61K 9/00; A61P 5/24

(52) U.S. Cl. CPC A61K 31/568; A61P 5/24; A61K 9/0019; A61K 47/34

(57) ABSTRACT

Disclosed herein are biodegradable poly(lactide-co-glycolide) (PLG) polymer compositions that are administered into the body with syringes or needles and that are utilized to deliver a testosterone into the body over an extended period of time.

### FIELD

This application pertains to the field of biodegradable polymer compositions that are administered into the body with syringes or needles and that are utilized to deliver testosterone into the body over an extended period of time.

### BACKGROUND

Hypogonadism is defined as deficient or absent male gonadal function, which results in insufficient testosterone secretion, or the failure to produce testosterone concentrations within a standard physiologic range and/or conduct normal spermatogenesis. Primary hypogonadism is due to testicular failure, which may be due to a congenital disorder such as Klinefelter's syndrome, or it may be due to an acquired disorder that may occur, for example, as a result of radiation treatment, chemotherapy, mumps, tumors, or trauma to the testes. Secondary hypogonadism is due to hypothalamic-pituitary axis dysfunction, resulting in the production or release of insufficient testosterone to maintain testosterone-dependent functions. In secondary hypogonadism a congenital or acquired disease state interferes with either the hypothalamus or the pituitary gland, the main glands that release hormones to stimulate the testes to produce testosterone. Hypogonadism can also result from a combination primary and secondary hypogonadism. Hypogonadism may occur at any age; however, low testosterone levels are more common in older males and this may result in infertility and sexual dysfunction. Hypogonadism may also increase the risk for depression, cardiovascular disease, type 2 diabetes, metabolic syndrome, and Alzheimer's disease.

Testosterone replacement therapy may produce a wide range of benefits for men with hypogonadism that include improvement in libido and sexual function, bone density, muscle mass, body composition, mood, erythropoiesis, cognition, and cardiovascular disease. Testosterone replacement therapy may also be used as a male contraceptive, or in transgender (female-to-male) hormone therapy. Testosterone replacement therapy may be administered orally, as a topical gel, transdermal patch, by injection, or as an implant surgically placed under the skin. Administration by injection or through an implant has the benefits of being able to provide a more consistent dose while having minimal risk of spreading testosterone to others. While testosterone therapy can provide a number of benefits for the patient (e.g., treating symptoms of hypogonadism) it can also negatively impact others, especially women and children, who come in contact with it.

There is a need in the art for a testosterone replacement formulation that safely, effectively, and consistently delivers a clinically effective amount of testosterone to a patient over an extended period of time.

### SUMMARY

In an aspect, the present disclosure provides a pharmaceutical composition, comprising: an active pharmaceutical ingredient (API) comprising testosterone or a pharmaceutically acceptable ester thereof; a solvent system comprising a biocompatible solvent and a low-molecular weight polyethylene glycol (PEG); and a biodegradable polymer comprising co-polymer segments of poly(lactide-co-glycolide) (PLG) and having at least one carboxylic acid end group.

In some embodiments, the active pharmaceutical ingredient is testosterone undecanoate.

In some embodiments, an amount of testosterone undecanoate in the composition is about 100 mg to about 400 mg per gram of the pharmaceutical composition.

In other embodiments, an amount of testosterone undecanoate in the composition is about 150 mg to about 250 mg per gram of the pharmaceutical composition.

In some embodiments, the active pharmaceutical ingredient is testosterone cypionate.

In some embodiments, an amount of testosterone cypionate in the composition is about 100 mg to about 400 mg per gram of the pharmaceutical composition.

In other embodiments, an amount of testosterone cypionate in the composition is about 150 mg to about 250 mg per gram of the pharmaceutical composition.

In some embodiments, the active pharmaceutical ingredient, prior to suspension in the pharmaceutical composition, has a D_{v,50} of about 1 μm to about 100 μm.

In other embodiments, the active pharmaceutical ingredient, prior to suspension in the pharmaceutical composition, has a D_{v,50} of about 30 μm to about 90 μm.

In yet other embodiments, the active pharmaceutical ingredient, prior to suspension in the pharmaceutical composition, has a D_{v,50} of about 35 μm to about 75 μm.

In some embodiments, the active pharmaceutical ingredient, prior to suspension in the pharmaceutical composition, has a D_{v,90} of about 100 μm to about 450 μm.

In other embodiments, the active pharmaceutical ingredient, prior to suspension in the pharmaceutical composition, has a D_{v,90} of about 300 μm to about 450 μm.

In some embodiments, the active pharmaceutical ingredient, prior to suspension in the pharmaceutical composition, has a span of about 1 to about 9.

In other embodiments, the active pharmaceutical ingredient, prior to suspension in the pharmaceutical composition, has a span of about 4 to about 9.

In yet other embodiments, the active pharmaceutical ingredient, prior to suspension in the pharmaceutical composition, has a span of about 1 to about 3.

In yet other embodiments, the active pharmaceutical ingredient, prior to suspension in the pharmaceutical composition, has a span of about 2 to about 7.

In some embodiments, the active pharmaceutical ingredient is milled to the target particle size distribution by dry milling, jet milling, nanomilling or wet milling in water or other solvent followed by lyophilization or drying, homogenization, ball milling, cutter milling, roller milling, grinding with a mortar and pestle, runner milling, cryomilling, or combinations thereof.

In some embodiments, the low molecular weight PEG comprises one or more PEGs having a number average molecular weight of about 3350 Daltons or less, and wherein an amount of the low molecular weight PEG is about 25 wt. % or less of the pharmaceutical composition.

In other embodiments, the amount of the low molecular weight PEG is about 15 wt. % or less of the pharmaceutical composition.

In yet other embodiments, the amount of the low molecular weight PEG is about 10 wt. % or less of the pharmaceutical composition.

In some embodiments, the low molecular weight PEG comprises terminal hydroxyl groups.

In other embodiments, the low molecular weight PEG comprises at least one end group selected from the group consisting of a hydroxyl group and a methyl ether group.

In some embodiments, the low molecular PEG is selected from the group consisting of PEG 250, PEG 300, PEG 350, PEG 400, PEG 600, PEG 1000, PEG 1450, and PEG 3350.

In some embodiments, the low molecular weight PEG is PEG 300.

In some embodiments, the low molecular weight PEG is PEG 400.

In some embodiments, the biocompatible solvent is selected from the group consisting of N-methyl-2-pyrrolidone (NMP), dimethyl sulfoxide (DMSO), polyethylene glycol (PEG), butyrolactone, N-cycylohexyl-2-pyrrolidone, diethylene glycol monomethyl ether, dimethyl acetamide, dimethyl formamide, ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, polyoxyl 35 hydrogenated castor oil, polyoxyl 40 hydrogenated castor oil, benzyl alcohol, n-propanol, isopropanol, tert-butanol, propylene glycol, 2-pyrrolidone, triacetin, tributyl citrate, acetyl tributyl citrate, acetyl triethyl citrate, triethyl citrate, an ester of any of the foregoing, and combinations of any of the foregoing.

In some embodiments, the biocompatible solvent is selected from the group consisting of N-methyl-2-pyrrolidone (NMP), dimethyl sulfoxide (DMSO), and a combination thereof.

In some embodiments, the biocompatible solvent comprises N-methyl-2-pyrrolidone (NMP).

In some embodiments, the biocompatible solvent system comprises N-methyl-2-pyrrolidone and PEG 300.

In some embodiments, the biodegradable polymer is formed with a hydroxy acid initiator.

In some embodiments, the hydroxy acid initiator is selected from the group consisting of GABA (gamma-amino butyric acid), GHB (gamma-hydroxybutyric acid), lactic acid, glycolic acid, citric acid, and undecylenic acid glycolic acid.

In some embodiments, the biodegradable polymer has a molar ratio of lactide to glycolide monomers of about 50:50 to about 90:10.

In other embodiments, the molar ratio of lactide to glycolide monomers is about to about 85:15.

In yet other embodiments, the molar ratio of lactide to glycolide monomers is about 70:30.

In yet other embodiments, the molar ratio of lactide to glycolide monomers is about 85:15.

In some embodiments, a weight average molecular weight of the biodegradable polymer is about 1 kDa to about 45 kDa.

In some embodiments, the weight average molecular weight of the biodegradable polymer is about 4 kDa to about 36 kDa.

In other embodiments, the weight average molecular weight of the biodegradable polymer is about 4 kDa to about 14 kDa.

In yet other embodiments, the weight average molecular weight of the biodegradable polymer is about 14 kDa to about 24 kDa.

In yet other embodiments, the weight average molecular weight of the biodegradable polymer is about 20 kDa to about 36 kDa.

In some embodiments, the active pharmaceutical ingredient makes up from about 10 wt % to about 30 wt % of the pharmaceutical composition.

In other embodiments, the active pharmaceutical ingredient makes up from about 15 wt % to about 25 wt % of the pharmaceutical composition.

In some embodiments, the solvent system makes up about 40 wt % to about 60 wt % of the pharmaceutical composition.

In other embodiments, the solvent system makes up about 45 wt % to about 55 wt % of the pharmaceutical composition.

In some embodiments, the biodegradable polymer makes up about 20 wt % to about 40 wt % of the pharmaceutical composition.

In other embodiments, the biodegradable polymer makes up about 25 wt % to about 35 wt % of the pharmaceutical composition.

In some embodiments, the active pharmaceutical ingredient makes up about 20 wt % of the composition, the biocompatible solvent system makes up about 50 wt % of the composition, and the biodegradable polymer makes up about 30 wt % of the pharmaceutical composition.

In some embodiments, the active pharmaceutical ingredient is in a substantially solid form in the pharmaceutical composition at temperatures up to about 35° C.

In some embodiments, the pharmaceutical composition has a viscosity of less than about 20,000 cP.

In other embodiments, the pharmaceutical composition has a viscosity of less than about 10,000 cP.

In yet other embodiments, the pharmaceutical composition has a viscosity of less than about 5,000 cP.

In an embodiment, the pharmaceutical composition comprises: about 20 wt % of testosterone undecanoate having a D_{v,50} of between about 35 μm to about 75 μm and a span of between about 2 to about 7, preferably the span is between about 2 to about 4 or between about 5 to about 7; about 50 wt % of a biocompatible solvent system comprising N-methyl-2-pyrrolidone (NMP) and polyethylene glycol with terminal hydroxyl groups having a number average molecular weight of about 300 Daltons (PEG 300), wherein a weight ratio of NMP to PEG 300 is about 4:1; and about 30 wt % of 70:30 poly(lactide-co-glycolide) (PLG) polymer having at least one carboxylic acid end group and having a weight average molecular weight of between about 4 kDa to about 24 kDa, preferably the weight average molecular weight is between about 4 kDa to about 14 kDa or between about 14 kDa to about 24 kDa.

In another embodiment, the pharmaceutical composition, comprises: about 20 wt % of testosterone undecanoate having a D_{v,50} of between about 35 μm to about 75 μm and a span of between about 2 to about 7; about 50 wt % of a biocompatible solvent system comprising N-methyl-2-pyrrolidone (NMP) and polyethylene glycol with terminal hydroxyl groups having a number average molecular weight of about 300 Daltons (PEG 300), wherein a weight ratio of NMP to PEG 300 is about 4:1; and about 30 wt % of 85:15 poly(lactide-co-glycolide) (PLG) polymer having at least one carboxylic acid end group and having a weight average molecular weight of between about 14 kDa to about 24 kDa.

In yet another embodiment, the pharmaceutical composition, comprises: about 20 wt % of testosterone cypionate having a D_{v,50} of between about 30 μm to about 50 μm and a span of between about 1 to about 3; about 50 wt % of a biocompatible solvent system comprising N-methyl-2-pyrrolidone (NMP) and polyethylene glycol with terminal hydroxyl groups having a number average molecular weight of about 300 Daltons (PEG 300), wherein a weight ratio of NMP to PEG 300 is about 3:2; and about 30 wt % of 70:30 poly(lactide-co-glycolide) (PLG) polymer and having at least one carboxylic acid end group and having a weight average molecular weight of between about 20 kDa to about 36 kDa.

Another aspect of the present disclosure is the use of the pharmaceutical composition disclosed herein as a medicament for testosterone replacement therapy for a condition associated with a deficiency or absence of endogenous testosterone.

In some embodiments, the condition is selected from the group consisting of primary hypogonadism and hypogonadotropic hypogonadism.

In some embodiments, the condition is congenital or acquired.

In some embodiments, the condition is female to male transgender.

Another aspect of the present disclosure is a solid depot formed upon administration of the pharmaceutical composition described herein into the body of a subject.

Another aspect of the present disclosure is to provide a product comprising the pharmaceutical composition described herein in the manufacture of a medicament for testosterone replacement therapy.

Another aspect of the present disclosure is a method of testosterone replacement therapy for a condition associated with a deficiency or absence of endogenous testosterone in a subject, comprising administering to the subject the pharmaceutical composition described herein.

In some embodiments, the pharmaceutical composition is administered subcutaneously.

In some embodiments, the pharmaceutical composition is administered once per about one month.

In other embodiments, the pharmaceutical composition is administered once per about two months.

In other embodiments, the pharmaceutical composition is administered once per about three months.

In yet other embodiments, the pharmaceutical composition is administered once per about four months.

In even yet other embodiments, the pharmaceutical composition is administered once per about five months.

In some embodiments, the pharmaceutical composition forms a solid in situ depot in a subject upon injection.

In some embodiments, the solid depot releases the active pharmaceutical ingredient in a clinically effective amount into the subject for at least about 30 days.

In other embodiments, the solid depot releases the active pharmaceutical ingredient into the subject for at least about 60 days.

In other embodiments, the solid depot releases the active pharmaceutical ingredient into the subject for at least about 90 days.

In yet other embodiments, the solid depot releases the active pharmaceutical ingredient into the subject for at least about 120 days.

In even yet other embodiments, the solid depot releases the active pharmaceutical ingredient into the subject for at least about 150 days.

In some embodiments, upon administering the pharmaceutical composition to a subject, an average serum testosterone concentration of the subject is about 3 ng/mL to about 10 ng/mL for at least about one month after administration.

In other embodiments, upon administering the pharmaceutical composition to a subject, the serum testosterone level of the subject is about 3 ng/mL to about 10 ng/mL for at least about two months after administration.

In other embodiments, upon administering the pharmaceutical composition to a subject, the serum testosterone level of the subject is about 3 ng/mL to about 10 ng/mL for at least about three months after administration.

In yet other embodiments, upon administering the pharmaceutical composition to a subject, the serum testosterone level of the subject is about 3 ng/mL to about 10 ng/mL for at least about four months after administration.

In even yet other embodiments, upon administering the pharmaceutical composition to a subject, the serum testosterone level of the subject is about 3 ng/mL to about 10 ng/mL for at least about five months after administration.

Another aspect of the present disclosure is a syringe comprising the pharmaceutical composition described herein.

In some embodiments, the syringe comprises a first chamber and a second chamber, wherein the pharmaceutical composition is stored in the first chamber and the second chamber is empty.

In some embodiments, the syringe comprises a first chamber and a second chamber, wherein the first chamber comprises the active pharmaceutical ingredient, wherein the second chamber comprises the solvent system and the biodegradable polymer.

In some embodiments, the pharmaceutical composition is mixed connecting the first and second chambers and then pushing the contents of the chambers between the first and second chambers.

In some embodiments, the syringe comprises a needle having a gauge of about 16 to about 22.

In some embodiments, the syringe comprises an injection volume of about 2 mL or less.

In other embodiments, the syringe comprises an injection volume of about 1 mL or less.

### BRIEF DESCRIPTION OF THE DRAWINGS

FIG. 1 shows the results of in vivo experiments comparing the mean testosterone concentration (ng/mL) in rats after injection with TU PLG copolymer formulations (Test Formulations 1 (♦), 2 (●), 3 (▪), 4 (⋄), 5 ( [symbol] ), 6 ( [symbol] ), and 7 ( )) and with a non-polymeric Control Formulation (○). The compositions of the TU PLG copolymer formulations and non-polymeric Control Formulation are provided in Tables 1 and 2.

FIG. 2 shows the results of in vivo experiments comparing the mean testosterone concentration (ng/mL) in rats after injection with TU PLG copolymer formulations. In FIG. 2A, Test Formulation 5 ( [symbol] ) has a weight average molecular weight of 9 kDa and Test Formulation 7 ( ) has a weight average molecular weight of 19 kDa. In FIG. 2B, Test Formulation 2 (●) has a weight average molecular weight of 9 kDa and Test Formulation 3 (▪) has a weight average molecular weight of 19 kDa. The compositions of the TU PLG copolymer formulations are provided in Table 1.

FIG. 3 shows the results of in vivo experiments comparing the mean testosterone concentration (ng/mL) in rats after injection with TU PLG copolymer formulations. In FIG. 3A, Test Formulation 1 (♦) comprises TU particles with a D_{v,50} of 4 and span of 2.3, Test Formulation 5 ( [symbol] ) comprises TU particles with a D_{v,50} of 53 and span of 6.2, and Test Formulation 2 (grey ●) comprises TU particles with a D_{v,50} of 67 and span of 6. In FIG. 3B, Test Formulation 4 (grey ⋄) comprises TU particles with a D_{v,50} of 18 and span of 2.7, Test Formulation 7 ( ) comprises TU particles with a D_{v,50} of 53 and span of 6.2, and Test Formulation 3 (▪) comprises TU particles with a D_{v,50} of 67 and span of 6. The compositions of the TU PLG copolymer formulations are provided in Table 1.

FIG. 4 shows the results of in vivo experiments comparing the mean testosterone concentration (ng/mL) in rats after injection with TU PLG copolymer formulations. Test Formulation 5 ( [symbol] ) has a L:G monomer molar ratio of 70:30 and Test Formulation 6 ( [symbol] ) has a L:G monomer molar ratio of 85:15. The compositions of the TU PLG copolymer formulations are provided in Table 1.

FIG. 5 shows the results of in vivo experiments comparing the mean testosterone concentration (ng/mL) in rats after injection with TU PLG copolymer formulation, Test Formulation 7, delivered in a low dose of 100 mg/kg (0.18 mL) (black - -, bottom line), a medium dose of 300 mg/kg (0.53 mL) (black dashed-dotted [symbol] , middle line), and a high dose of 500 mg/kg (0.88 mL) (grey dashed-dotted [symbol] , top line). The composition of Test Formulation 7 is provided in Table 1.

FIG. 6 shows the results of in vivo experiments comparing the mean testosterone concentration (ng/mL) in rats after injection with TU PLG copolymer formulations Test Formulation 3 (▪) comprises TU particles with a D_{v,50} of 67 μm, a D_{v,90} of 412 μm, and a span of 6, Test Formulation 7 ( ) comprises TU particles with a D_{v,50} of 53 μm, a D_{v,90} of 340 μm, and a span of 6.2, and Test Formulation 8 (♦) comprises TU particles with a D_{v,50} of 51 μm, a D_{v,90} of 146 μm, and a span of 2.6. The compositions of the TU PLG copolymer formulations are provided in Tables 1 and 5.

FIG. 7 shows the results of in vivo experiments comparing the mean testosterone concentration (ng/mL) in rats after injection with TC PLG copolymer formulations (Test Formulations 9 (dashed [symbol] ), 10 ( [symbol] ), and 11 (grey dashed-dotted [symbol] ). The compositions of the TC PLG copolymer formulations are provided in Table 7.

FIG. 8 shows the results of in vivo experiments comparing the mean testosterone concentration (ng/mL) in rats after injection with a TC PLG copolymer formulation (Test Formulation 9 (★); see Table 7 for the formulation composition). For comparison, the release profiles of TU PLG copolymer formulation (Test Formulations 3 (▪) and 4 (⋄); see Table 1 for the formulation compositions) are also shown.

FIG. 9 shows the results of in vivo experiments comparing the mean testosterone concentration (ng/mL) in minipigs after injection with TU PLG copolymer formulation (Test Formulations 12 (black solid [symbol] , upper line) and 13 (grey dashed-dotted [symbol] , bottom line)). The compositions of the TU PLG copolymer formulations are provided in Table 9.

FIG. 10 shows the TU in vivo release profile for minipigs receiving a low dose of 20 mg/kg (1× 1 mL) (black -▴-, bottom line), a medium dose of 90 mg/kg (3× 1.5 mL) (grey [symbol] , middle line), and a high dose of 160 mg/kg (4× 2 mL) (dashed [symbol] , top line) of Test Formulation 14. The compositions of the TU PLG copolymer Test Formulation 14 is provided in Table 11.

FIG. 11 shows the results of in vitro experiments comparing the mean testosterone undecanoate release achieved with TU PLG copolymer Test Formulations 15 ( [symbol] ), 16 (--+--), and 17 (-X-), shown as the cumulative TU release. The compositions of the TU PLG copolymer formulations are provided in Table 13.

### DETAILED DESCRIPTION

Described herein are pharmaceutical compositions that can be administered into the body of a subject or patient via syringes or needles for the release of testosterone or a pharmaceutically acceptable ester thereof (or suitable analog thereof) over an extended period of time. The compositions described herein can deliver consistent levels of testosterone or an ester thereof within a therapeutic window to a patient for extended periods of time. In particular, the present disclosure is directed to extended release pharmaceutical compositions, which include a biodegradable polymer comprising co-polymer segments of poly(lactide-co-glycolide) (PLG), a biocompatible solvent system comprising a biocompatible solvent and at least one low-molecular weight polyethylene glycol (PEG), and testosterone or a pharmaceutical acceptable ester thereof suspended therein. The pharmaceutical compositions may be used to provide a biodegradable (or bioerodible) in situ formed solid implant or depot in a subject. The composition is administered as a flowable suspension into tissue and a solid depot is formed in situ upon dissipation of the solvent. The depot is used to deliver testosterone or a pharmaceutically acceptable ester thereof in a controlled, or extended, release manner to a subject over a period of about 30 days to about 180 days, or over a period of about 60 days to about 150 days, or over a period of about 60 days to about 120 days, or over a period of about 60 days to about 90 days, or over a period of about 90 days. The pharmaceutical compositions of the present disclosure are the result of particular, novel and inventive combinations of: (1) polymer type, molecular weight ranges, and monomer ratio ranges; (2) solvent types and ranges; and/or (3) drug forms and drug substance particle size ranges, that together, provide formulations that deliver predictable testosterone levels over the extended treatment period, resulting in the desired target serum testosterone concentrations in a patient.

The pharmaceutical compositions disclosed herein comprise testosterone or a pharmaceutically acceptable ester thereof (or suitable analog thereof, which may include a salt or derivative of testosterone or an ester thereof) as an active pharmaceutical ingredient (API), which can be generally referred to herein as a “testosterone API”. Suitable testosterone API's for use in the present disclosure will preferably be in a stable suspension in a formulation of the present disclosure (i.e., when combined with the biodegradable polymer and solvent system as described herein). In embodiments, a preferred testosterone API is selected from testosterone or an ester of testosterone. Examples of suitable esters of testosterone for use in the present disclosure include testosterone undecanoate (TU) which is also known as testosterone undecylate, testosterone cypionate (TC), testosterone propionate, and testosterone busciclate. Testosterone undecanoate, testosterone cypionate, testosterone propionate, and testosterone busciclate are prodrugs of the hormone testosterone; they are esters of testosterone used in androgen replacement therapy, primarily for the treatment of male hypogonadism. Testosterone API's provided within a formulation of the present disclosure may also be used as a male contraceptive, or in transgender (female-to-male) hormone therapy. Use of a prodrug of testosterone, e.g., a testosterone ester, may provide advantages or benefits in certain applications. For example, it may improve the stability of the formulation (e.g., during storage or irradiation, or after delivery in vivo), delay the release of the active form of the drug, affect or modify the solubility of the drug in the formulation, and/or extend or otherwise modify the duration of action of the drug.

The pharmaceutical compositions of this disclosure, which may also be referred to as controlled release compositions (or formulations) or extended release compositions (or formulations), are used to provide a biodegradable or bioerodible in situ formed depot in a subject. The biodegradable polymers or copolymers used herein are substantially insoluble in water and body fluid. The compositions, prior to administration and at the moment of administration, are flowable compositions composed of: (1) a biodegradable polymer or copolymer, and particularly a biodegradable thermoplastic polymer comprising co-polymer segments of poly(lactide-co-glycolide) (PLG) and having at least one carboxylic acid end group; in combination with (2) a suitable solvent system comprising a biocompatible solvent and a co-solvent of at least one low-molecular weight polyethylene glycol (PEG); and (3) a testosterone API which is preferably testosterone or a pharmaceutically acceptable ester thereof, suspended therein. The flowable, extended release composition is administered as a liquid or gel into tissue, wherein a solid depot forms in situ upon dissipation of the solvent.

As used herein, “flowable” refers to the ability of the composition to be injected through a medium (e.g., syringe) into the body of a subject. For example, the composition can be injected, with the use of a syringe, beneath the skin of a subject (i.e., subcutaneously) or into the muscle (i.e., intramuscularly). The ability of the composition to be injected into a subject will typically depend upon the viscosity of the composition, and the device used (i.e., type of device, whether manual or auto, needle gauge, etc.). The composition should therefore have a suitable viscosity prior to injection, such that the composition can be forced through the medium (e.g., syringe) into the body of a subject, yet the composition should still be sufficiently viscous such that the API remains suspended in the composition prior to injection. Typically, the viscosity of the composition is between about 500 cP and about 20,000 cP, or between about 500 cP and about 10,000 cP, or between about 500 cP and about 5,000 cP, or between about 500 cP and about 3,000 cP, or between about 500 cP and about 1,500 cP, or between about 1500 cP and about 3000 cP, or between about 2000 cP and about 2500 cP, or it may be less than about 20,000 cP, or less than about 10,000 cP, or less than about 5,000 cP, or less than about 3000 cP. The viscosity of the composition may be such that the composition may be administered by manual injection though a syringe with, for example, a 16 to 24 gauge needle, or an 18 to 22 gauge needle, or an 18 to 20 gauge needle, or may be administered by injection using an autoinjector.

As discussed above, upon injection of the extended release composition into a subject, the solvent dissipates, and an in-situ solid depot is formed. The polymer depot degrades by hydrolysis until the remaining polymer fragments are small enough to diffuse out of the depot. During degradation, the testosterone API is released from the depot over an extended time period. The depot, so formed, is optimally used to consistently deliver therapeutic amounts of the testosterone API in a controlled, or extended, release manner to the subject over a dosing period of about 30 days to about 180 days, or over a period of about 60 days to about 150 days, or over a period of about 60 days to about 120 days, or over a period of about 60 days to about 90 days, or about 30 days, about 60 days, about 90 days, about 120 days, about 150 days, or about 180 days (or longer). The extended release composition, on average during the dosing period, provides testosterone supplementation that achieves target serum testosterone concentrations that are reflective of that in healthy males (e.g., in the eugonadal range). Specifically, in one embodiment, and as an illustrative example, at least 75% of patients have an average concentration of testosterone in plasma (C_{avg}) of 10.4 nmol/L to 34.7 nmol/L (i.e., 3 ng/mL to 10 ng/mL), wherein the lower limit of the 95% confidence interval for the percentage of subjects with C_{avg} within the eugonadal range is ≥65%, and at no point during the dosing period does the maximum concentration of testosterone in plasma (Cₘₐₓ) exceed 25 ng/mL, and 5% or less of patients have a Cₘₐₓ of between 18 ng/mL and 25 ng/mL and greater than 85% of patients have a Cₘₐₓ of less than 15 ng/mL (see, e.g., Shehzad Basaria, “Male hypogonadism,” 383 Lancet 1250 (2014); Abraham Morgentaler et al., “Long acting testosterone undecanoate therapy in men with hypogonadism: results of a pharmacokinetic clinical study,” 180 J. Urology 2307 (2008)).

A beneficial characteristic of the compositions disclosed herein is the ability for the compositions to provide for extended release of a therapeutically effective amount of testosterone to a subject. As such the amount of the testosterone API present in the composition should be sufficient to achieve the desired therapeutic effect, e.g., to, on average, provide testosterone supplementation in the eugonadal range (e.g., 3 ng/mL to 10 ng/mL testosterone in plasma, or a broader range or overlapping range, if therapeutically effective) to treat or reduce the symptoms of androgen deficiency; to treat or reduce the symptoms of male hypergonadism; as an adjunct therapy for transgender men or gender reassignment; or as birth control. In addition, the amount of testosterone API present in the composition should be suitable for long term treatment in accordance with the time frames disclosed herein. For example, a single dosage formulation can include sufficient amounts of testosterone API for treatment of a patient for at least one week, at least two weeks, for at least one month, for at least two months, for at least three months, for at least four months, for at least five months, or for at least six months.

### Biodegradable Polymer

As used herein, the term “polymer” may be defined as a macromolecular organic compound that is largely, but not necessarily exclusively, formed of repeating units covalently bonded in a chain, which may be linear or branched. A “repeating unit” is a structural moiety of the macromolecule that can be found within the macromolecular structure more than once. Typically, a polymer is composed of a large number of a few types of repeating units that are joined together by covalent chemical bonds to form a linear backbone, from which substituents may or may not depend in a branching manner. The repeating units can be identical to each other but are not necessarily so. Therefore, a structure of the type -A-A-A-A- wherein A is a repeating unit is a polymer is known as a homopolymer. Whereas, a structure of the type -A-B-A-B- or -A-A-A-B-A-A-A-B- wherein A and B are repeating units, is also a polymer, and is sometimes termed a copolymer. A structure of the type -A-A-A-C-A-A-A or A-B-A-C-A-B-A wherein A and B are repeating units but C is not a repeating unit (i.e., C is found once within the macromolecular structure) is also a polymer under the definition herein. When C is flanked on both sides by repeating units, C is referred to as a “core” or a “core unit.” A short polymer, formed of up to about 10 repeating units, is referred to as an “oligomer.” There is theoretically no upper limit to the number of repeating units in a polymer, but practically speaking the upper limit for the number of repeating units in a single polymer molecule may be approximately one million. However, in the polymers of the present disclosure the number of repeating units is typically in the hundreds or less. In some embodiments, the term “polymer” may be used interchangeably with the term “biodegradable polymer”.

The term “copolymer” may be used to refer to a variety of polymers comprising non-identical repeating units. A “copolymer” may be regular or random in the sequence as defined by the more than one type of repeating unit. Some types of copolymers are random copolymers, graft copolymers and block copolymers.

The term “polymer segment” or a “copolymer segment” as used herein may refer to a portion or moiety of a larger molecule wherein that segment is a section of a polymer or a copolymer respectively that is bonded to other portions or moieties to make up the larger molecule. When the polymer segment or a copolymer segment is attached to the larger molecule at one end of the segment, the end of attachment is the “proximal end” and the other, free end is the “distal end.”

As used herein, the term “biodegradable” refers to any water-insoluble material that is converted under physiological conditions into one or more water-soluble materials, without regard to any specific degradation mechanism or process. The term “bioerodible” refers to any water-insoluble material that is converted under physiological conditions into one or more water-soluble materials with or without changes to the chemical structure.

The biodegradable polymer used in the compositions of the present disclosure is a poly(lactide-co-glycolide) polymer, and preferably a poly(D,L-lactide-co-glycolide) polymer. The PLG polymer is typically formed by ring-opening polymerization from lactide and glycolide monomers. The term “poly(lactide-glycolide)”, “poly(lactide-co-glycolide)”, or “PLG” may be used interchangeably herein to refer to a copolymer or a copolymer segment formed of dimeric units of lactic acid and dimeric units of glycolic acid that make up the polymeric chain. A PLG polymer is typically formed through polymerization of the cyclic dimers lactide and glycolide, although it could also be theoretically formed through any process wherein dimeric units are incorporated in a given step of the polymerization process. The PLG polymers of the present disclosure are solid polymers and form solid depots within the body, meaning that the melting temperature of the polymer is above body temperature (e.g., about 36.5° C. to about 37.5° C. (about 97.7° F. to about 99.5° F.)).

As used herein, the term “lactide” may be used herein, when referring to the chemical compound itself, for example as the “lactide reagent” or “lactide reactant”, means the dimer cyclic ester of lactic acid:

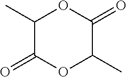

Lactide may be of any configuration at the chiral carbon atoms (bearing the methyl groups). It may also be a mixture of molecules with different configurations at the chiral carbon atoms. Thus, lactide may be DD-, DL-, LD-, LL-lactide, or any mixture or combination thereof. In some embodiments, when referring to a polymer such as a “poly(lactide-co-glycolide)’ containing a “lactide” unit, the term “lactide” or “lactide unit” means the ring-opened species consisting of two lactic acid units joined by an ester bond which can be further incorporated into a polymeric chain with other such units or with other types of repeating units. One end of the lactide unit comprises a carboxyl group that may be bonded to an adjacent atom via an ester linkage, or an amide linkage, or via any other type of bond that a carboxyl group may form. The other end of the lactide unit comprises a hydroxyl group that may be bonded to an adjacent atom via an ester linkage, an ether linkage, or via any other type of bond that a hydroxyl group may form. A “lactide” in a poly-lactide polymer thus refers to the repeating unit of the polymer that can be viewed structurally as being formed from a pair of lactic acid molecules, with the understanding that the wavy lines indicate points of attachment to neighboring groups:

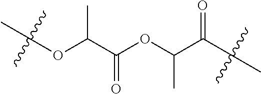

The configuration at the chiral carbon atoms includes any and all possible configurations and mixtures thereof, as described above for the cyclic dimer. Polylactide exists in two stereo forms, signified by a D or L for dextrorotatory or levorotatory, or by DL for the racemic mix, e.g., poly(D,L-lactide) or poly(D,L-lactide-co-glycolide).

The term “glycolide” may be used herein, when referring to the chemical compound itself, such as the “glycolide reagent” or the “glycolide reactant”, means the dimer cyclic ester of glycolic acid:

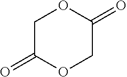

When referring to a “glycolide” unit in a polymer, the term refers to the repeating unit, a dimer of glycolic acid as shown:

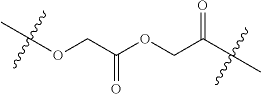

Similarly to the lactide unit, in some embodiments, one end of the glycolide unit may comprise a carboxyl group bonded to an adjacent atom via an ester linkage, or an amide linkage, or via any other type of bond that a carboxyl group may form, and the other end of the glycolide unit comprises a hydroxyl group that may be bonded to an adjacent atom via an ester linkage, an ether linkage, or via any other type of bond that a hydroxyl group may form.

In some embodiments, the PLG polymer has at least one carboxylic acid end group. The at least one carboxylic acid end group is not protected; it is not in the form of ester or any other functional group that serves as a protecting group to a carboxylic acid. Typically, a PLG polymer with an acid end group is made by the ring opening polymerization of lactide and/or glycolide monomers, by standard chain-growth polymerization techniques, which is initiated by water or a carboxylic acid compound of the formula Nu—R—COOH where Nu is a nucleophilic moiety, such as an amine or hydroxyl, R is any organic moiety, and the —COOH is a carboxylic acid functionality. The nucleophilic moiety of the molecule acts to initiate the ring opening polymerization in the presence of a catalyst and heat, producing a polymer with a carboxylic acid functionality on one end. Carboxylic acids that are suitable initiators are those that contain an alkyl chain, a nucleophile, and are soluble in the solvent used to make the polymer. Examples of suitable initiators include, but are not limited to, GABA (gamma-amino butyric acid), GHB (gamma-hydroxybutyric acid), lactic acid, glycolic acid, citric acid, and undecylenic acid. Alternatively, a carboxylic acid end group may be created on the end of a polymer chain by post-polymerization modification. The presence of the carboxylic acid end group on the polymer increases the hydrophilicity of the polymer, compared to PLG polymers with other end groups such ester and/or hydroxy groups, and can influence the degradation of the polymer and the release of the API in situ.

As used herein, the term “catalyst”, may refer to any suitable substance capable of initiating or and/or increasing the rate of polymerization. In some embodiments, the catalyst may be any catalyst suitable for ring-opening polymerization. For example, a tin salt of an organic acid may be used as the polymerization catalyst. The tin salt may be either in the stannous (divalent) or stannic (tetravalent) form. In some instances, the catalyst may be stannous octanoate. The catalyst may be present in the polymerization reaction mixture in any suitable amount, typically ranging from about 0.01 to 1.0 percent.

In embodiments, the biodegradable copolymer has a molar ratio of lactide to glycolide (L:G) monomers of any two whole numbers X to Y (i.e., X:Y), where Xis at least about 50 and no more than about 90 and the sum of X and Y is 100. Unless otherwise specified, all ratios between monomers in a copolymer disclosed herein are molar ratios. In other words, in embodiments, the PLG copolymer has a molar ratio of lactide to glycolide monomers from about 50:50 to about 90:10. In some embodiments, the PLG copolymer has a molar ratio of lactide to glycolide monomers from about 70:30 to about 85:15. In some embodiments, the PLG copolymer has a molar ratio of lactide to glycolide monomers monomer units of about 70:30, or about 75:25, or about 80:20, or about 85:15.

In embodiments, the PLG copolymer may optionally be purified prior to use in the extended-release formulation to remove low-molecular weight oligomers and/or unreacted monomers and/or catalyst. Several methods of purifying polymers are known in the art, including the methods described in U.S. Pat. Nos. 4,810,775, 7,019,106, and 9,187,593, among others.

As used herein, the terms “molecular weight” and “average molecular weight,” unless otherwise specified, mean a weight-average molecular weight as measured by a conventional gel permeation chromatography (GPC) instrument (such as an Agilent 1260 Infinity Quaternary LC with Agilent G1362A Refractive Index Detector) utilizing polystyrene standards and tetrahydrofuran (THF) as the solvent. Furthermore, the “molecular weight” and “average molecular weight” and “weight-average molecular weight” reported herein, unless otherwise specified, refers to the molecular weight of the polymer or copolymer within the extended release composition, after the composition has undergone sterilization by electron beam (e-beam) irradiation. It is well-known that process of e-beam sterilization reduces the molecular weight of the polymer due to breakage of the polymer bonds, leading to a shorter polymer chain with lower MW. The amount of MW decrease due to e-beam irradiation has been characterized and is accounted for during manufacturing, and can be, for example, about 0.1-25% depending upon the initial size of the polymer. The ranges “molecular weight” and “average molecular weight” and “weight average molecular weight” reported herein may also refer to the molecular weight ranges for the release specifications of the polymer.

In some embodiments, a weight average molecular weight of the biodegradable polymer may be from about 1 kDa to about 45 kDa, or from about 4 kDa to about 40 kDa, or from about 4 kDa to about 36 kDa, or from about 4 kDa to about 30 kDa, or from about 4 kDa to about 25 kDa, or from about 4 kDa to about 24 kDa, or from about 4 kDa to about 23 kDa, or from about 4 kDa to about 22 kDa, or from about 4 kDa to about 21 kDa, or from about 4 kDa to about 20 kDa, or from about 4 kDa to about 19 kDa, or from about 4 kDa to about 18 kDa, or from about 4 kDa to about 17 kDa, or from about 4 kDa to about 16 kDa, or from about 4 kDa to about 15 kDa, or from about 4 kDa to about 14 kDa, or from about 4 kDa to about 13 kDa, or from about 4 kDa to about 12 kDa, or from about 4 kDa to about 11 kDa, or from about 4 kDa to about 10 kDa, or alternatively any whole number to any other whole number from 1 kDa to about 45 kDa. In some embodiments, a weight average molecular weight of the biodegradable polymer may be from 14 kDa to about 40 kDa, or from about 14 kDa to about 36 kDa, or from about 14 kDa to about 30 kDa, or from about 14 kDa to about 25 kDa, or from about 14 kDa to about 24 kDa, or from about 14 kDa to about 23 kDa, or from about 14 kDa to about 22 kDa, or from about 14 kDa to about 21 kDa, or from about 14 kDa to about 20 kDa. In some preferred embodiments, a weight average molecular weight of the biodegradable polymer may be about 4 kDa to about 14 kDa; yet in other preferred embodiments, a weight average molecular weight of the biodegradable polymer may be about 14 kDa to about 24 kDa; while yet in other preferred embodiments, a weight average molecular weight of the biodegradable polymer may be about 20 kDa to about 36 kDa. In some embodiments of the composition, the biodegradable polymer has a weight average molecular weight of about 4 kDa, or about 5 kDa, or about 6 kDa, or about 7 kDa, or about 8 kDa, or about 9 kDa, or about 10 kDa, or about 11 kDa, or about 12 kDa, or about 13 kDa, or about 14 kDa, or about 15 kDa, or about 16 kDa, or about 17 kDa, or about 18 kDa, or about 19 kDa, or about 20 kDa, or about 21 kDa, or about 22 kDa, or about 23 kDa, or about 24 kDa, or about 25 kDa, or about 26 kDa, or about 27 kDa, or about 28 kDa, or about 29 kDa, or about 30 kDa, or about 31 kDa, or about 32 kDa, or about 33 kDa, or about 34 kDa, or about 35 kDa, or about 36 kDa. In preferred embodiments, the biodegradable polymer has a weight average molecular weight of about 9 kDa; while in other preferred embodiments the biodegradable polymer has a weight average molecular weight of about 19 kDa; while yet in other preferred embodiments the biodegradable polymer has a weight average molecular weight of about 28 kDa.

In some embodiments, the biodegradable polymer may be a poly(lactide-co-glycolide) copolymer comprising a lactide to glycolide monomer molar ratio from about 70:30 to about 85:15, wherein the polymer has at least one carboxylic acid end-group and a weight average molecular weight from about 4 kDa to about 36 kDa. In some preferred embodiments, the biodegradable polymer may be a poly(lactide-co-glycolide) copolymer comprising a lactide to glycolide monomer molar ratio of about 70:30 or about 85:15, wherein the polymer has at least one carboxylic acid end-group and a weight average molecular weight from about 4 kDa to about 14 kDa, or more preferably a weight average molecular weight of about 9 kDa. In other preferred embodiments, the biodegradable polymer may be a poly(lactide-co-glycolide) copolymer comprising a lactide to glycolide monomer molar ratio of about 70:30 or about 85:15, wherein the polymer has at least one carboxylic acid end-group and a weight average molecular weight from about 14 kDa to about 24 kDa, or more preferably a weight average molecular weight of about 19 kDa. In yet other preferred embodiments, the biodegradable polymer may be a poly(lactide-co-glycolide) copolymer comprising a lactide to glycolide monomer molar ratio of about 70:30 or about 85:15, wherein the polymer has at least one carboxylic acid end-group and a weight average molecular weight from about 20 kDa to about 36 kDa, or more preferably a weight average molecular weight of about 28 kDa.

The biodegradable polymer may make up from about 10 wt % to about 50 wt % of the composition, or preferably from about 20 wt % to about 40 wt % of the composition, or more preferably from about 25 wt % and about 35 wt % of the composition, or even more preferably about 30 wt % of the composition. Alternatively, the biodegradable polymer may make up any whole-number weight percentage of the composition between about 10 wt % and about 50 wt %, or may make up a range from any whole-number weight percentage of the composition to any other whole-number weight percentage of the composition from about 10 wt % to about 50 wt %.

### Biocompatible Solvent System

The extended release composition of the present disclosure comprises a biocompatible solvent system that is capable of dissolving the biodegradable polymer and forming a suspension with the testosterone API, when the three components are combined, and also dissipates within the body to enable the formation of the solid depot in situ. The solvent system comprises at least one biocompatible solvent, and at least one low molecular weight polyethylene glycol (PEG) as a co-solvent. The solvent system partially or completely dissipates or diffuses into host surrounding tissues upon administration thereof. Diffusion or dissipation of the solvent system upon administration into bodily fluids allows for solidification of the polymer and the testosterone API suspended therein as a solid depot via coagulation or precipitation of both components within bodily fluids. The extent of water insolubility of the solvents and co-solvents in the solvent system impact the desired rate of diffusion into bodily fluids for controlling the rate and scope of polymer solidification. Furthermore, the solvent/co-solvents control the viscosity of the flowable extended release composition, which aids in preparing and administering the extended release composition to a subject. The formulations of the present disclosure provide inventive solvent systems which help to achieve the desired formulation characteristics and extended release profiles described herein.

As used herein, the term “solvent” refers to a liquid that dissolves a solid or liquid solute, or to a liquid external phase of a suspension throughout which solid particles are dispersed. The term “co-solvent” refers to a substance added to a solvent to modify the solubility of a solute in the solvent. The term “solvent system” as used herein refers to the combination of at least one biocompatible solvent as described herein and at least one low molecular weight PEG as a co-solvent. As used herein, the term “biocompatible solvent” may be defined as any solvent safe for injection within a human body. The term “biocompatible solvent” may be used interchangeably with to the term, “solvent”. The biocompatible solvent may be a mixture of solvents and/or co-solvents and may be homogenous or heterogeneous in nature. The solvent may be an organic solvent (carbon-based) and may further be a polar aprotic solvent which is generally non-toxic in bodily fluids. The solvent may be partially to completely water-insoluble.

Biocompatible solvents and co-solvents suitable for use in embodiments of the present disclosure include or may be at least partially made up of one or more solvents selected from the group consisting of amides, acids, alcohols, esters of monobasic acids, ether alcohols, sulfoxides, lactones, polyhydroxy alcohols, esters of polyhydroxy alcohols, ketones, and ethers. Suitable solvents and co-solvents of the present disclosure include, by way of non-limiting example, N-methyl-2-pyrrolidone (NMP), dimethyl sulfoxide (DMSO), polyethylene glycol (PEG), butyrolactone, N-cycylohexyl-2-pyrrolidone, diethylene glycol monomethyl ether, dimethyl acetamide, dimethyl formamide, ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, polyoxyl 35 hydrogenated castor oil, polyoxyl 40 hydrogenated castor oil, benzyl alcohol, n-propanol, isopropanol, tert-butanol, propylene glycol, 2-pyrrolidone, triacetin, tributyl citrate, acetyl tributyl citrate, acetyl triethyl citrate, triethyl citrate, an ester of any of the foregoing, and combinations of any of the foregoing. In some embodiments, the biocompatible solvent comprises at least one of N-methyl-2-pyrrolidone and dimethyl sulfoxide. In preferred embodiments, the biocompatible solvent comprises N-methyl-2-pyrrolidone.

Where the solvent is a combination or mixture of solvents and/or co-solvents, any two of the solvents and/or co-solvents in the mixture may be present in any weight ratio between about 1:99 and about 99:1. Where the solvent comprises two or more solvents and/or co-solvents, any two of them may be present in any weight ratio between about 99:1 and about 1:99, or between about 90:10 and about 10:90, or between about 80:20 and about 20:80, or between about 30:70 and about 70:30, or between about 40:60 and about 60:40, or about 50:50, or alternatively in any weight ratio X:Y where each of X and Y is a whole number between about 1 and about 99 and the sum of X and Y is 100.

As discussed above, the solvent system used in the present disclosure comprises, in addition to the at least one biocompatible solvent, a co-solvent in the form of one or more low molecular weight polyethylene glycols (PEGs). The use of PEG as a co-solvent in the solvent system herein improves the degree to which the testosterone API is suspended in the formulation. The low molecular weight PEG is a biocompatible solvent that acts as a liquid carrier and solvates the polymer. Because it generally limits the solubility of testosterone esters such as TU in the formulation, the addition of the low molecular weight PEG improves the thermal stability of the formulation, leading to a more controlled manufacturing process and final product.

Typically, the low molecular weight PEGs utilized in the present disclosure have a number average molecular weight of about 3350 Daltons or less (e.g., PEG 3350 or less, wherein the PEG decreases in number average molecular weight by an integer number value of 44 g/mol which represents a single ethylene glycol (EG) monomer). As is known in the art, reference to a PEG co-solvent of a particular number average molecular weight, generally refers to a material that is not mono-disperse; i.e., there is a distribution of PEG moieties within the material that together, provide an average molecular weight of the target molecular weight. For example, PEG 300 is a mixture of PEG moieties with a molecular weight distribution that results in a number average molecular weight of 300 Da (300 Da is the target molecular weight). Accordingly, PEG 300 means PEG with a number average molecular weight of 300 Da; PEG 400 means PEG with a number average molecular weight of 400 Da, and so on.

As is known in the art, PEG may be linear or branched. PEG typically refers to poly(ethylene glycol) with terminal hydroxyl (—OH) groups, but alternate or derivative forms of PEG exist that have one or more different end groups other than hydroxyl groups. For example, poly(ethylene glycol) monomethyl ether has one terminal hydroxyl (—OH) and one terminal methyl ether (—CH₃) group, while poly(ethylene glycol) dimethyl ether has two terminal methyl ether (—CH₃) groups. As used herein “PEG” refers to a polymer having repeating ethylene glycol (EG) monomers; the end groups may be hydroxyl (—OH) groups or another chemical moiety. Suitable PEG end groups may include, but are not limited to, hydroxyl, methyl ether, methyl ester, acrylate, methacrylate, maleimide, vinyl sulfonate, norbornene, N-hydroxysuccinimide ester, aldehyde, anhydride, epoxide, isocyanate, sulfonyl chloride, fluorobenzene, imidoester, carbodiimide, acyl azide, carbonate, fluorophenyl ester, thiol, amine, carboxyl, and carbonyl. In some embodiments, the low-molecular weight PEG comprises at least one end group selected from the group consisting of a hydroxyl group and a methyl ether group. In preferred embodiments, the low-molecular weight PEG comprises terminal hydroxyl groups.

In some embodiments, a single low molecular weight PEG may be included in the solvent system. In other embodiments, two low molecular weight PEGs may be included in the solvent system. In other embodiments, three or more low molecular weight PEGs may be included in the solvent system. Suitable low molecular weight PEGs useful in the present disclosure may include, but are not limited to, PEG 300, PEG 400, PEG 500, PEG 600, PEG 1000, PEG 1450, and PEG 3350. These PEGs have terminal hydroxyl groups. Other suitable low molecular weight PEGs may include, for example, PEG 250 and PEG 350. PEG 250 may have terminal methyl ether groups and PEG 350 may have a terminal methyl ether group and a terminal hydroxyl group. In some embodiments, the low molecular weight PEG has a number average molecular weight of about 1000 Daltons or less (i.e., PEG 1000 or less). In some embodiments, the low molecular weight PEG has a number average molecular weight of about 600 Daltons or less (i.e., PEG 600 or less). In some embodiments, the low molecular weight PEG is PEG 300 or PEG 400.

In some embodiments, the low molecular weight PEG may be present in an amount from about 25 wt % or less, or about 20 wt % or less, or about 15 wt % or less, or about 10 wt % or less. In some embodiments the low molecular weight PEG may be present in an amount from about 1 wt % to about 25 wt %, or from about 1 wt % to about 20 wt %, or from about 1 wt % to about 15 wt %, or from about 1 wt % to about 10 wt %, or from about 1 wt % to about 9 wt %, or from about 1 wt % to about 8 wt %, or from about 1 wt % to about 7 wt %, or from about 1 wt % to about 6 wt %, or from about 1 wt % to about 5 wt %, or from about 1 wt % to about 4 wt %, or from about 1 wt % to about 3 wt %, or from about 1 wt % to about 2 wt % of the formulation, or from about 5 wt % to about 15 wt %, or from about 6 wt % to about 14 wt %, or from about 7 wt % to about 13 wt %, or from about 8 wt % to about 12 wt %, or from about 9 wt % to about 11 wt %, or about 10 wt %, or alternatively as any whole number percentage by weight of the formulation from about 1 wt % to about 25 wt %, both inclusive. Typically, as a general non-limiting observation, when a smaller low-molecular weight PEGs (e.g., PEG 250, PEG 300, PEG 350, PEG 400, and PEG 600) is included in the solvent system, the amount of the low-molecular weight PEG will be greater than when a larger low-molecular weight PEGs (e.g., PEG 3350 and PEG 1450) is included in the solvent system. The amount of low molecular weight PEG present in the formulation is such that it improves the thermal stability of the formulation; however, the formulations disclosed herein also remain flowable (i.e., suitable for injection) at room temperature and at refrigeration temperatures (i.e., 0-8° C.).

In embodiments of the present disclosure, the solvent system may be present in any amount between about 30 wt % and about 70 wt % of the composition, or between about 40 wt % and about 60 wt % of the composition, or between about 45 wt % and about 55 wt % of the composition, or about 50 wt % of the composition, or alternatively the solvent system can range from any whole number percentage by weight of the composition to any other whole number percentage by weight of the composition between about 40 wt % and about 70 wt %.

In some embodiments, the solvent system may be a mixture of NMP and low molecular weight PEG, preferably PEG 300 or PEG 400, where the weight ratio of NMP to low molecular weight PEG is between about 1:1 to about 5:1, both inclusive, or is about 1:1, or about 1.5:1, or about 2:1, or about 2.5:1, or about 3:1, or about 3.5:1, or about 4:1, or about 4.5:1, or about 5:1. In other embodiments, the solvent system may be a mixture of DMSO and a low molecular weight PEG, preferably PEG 300 or PEG 400, where the weight ratio of DMSO to low molecular weight PEG is between about 1:1 to about 5:1, both inclusive, or is about 1:1, or about 1.5:1, or about 2:1, or about 2.5:1, or about 3:1, or about 3.5:1, or about 4:1, or about 4.5:1, or about 5:1.

### Active Pharmaceutical Ingredient

The pharmaceutical compositions disclosed herein comprise testosterone or a pharmaceutically acceptable ester thereof (or suitable analog thereof, which may include a salt or derivative of testosterone or ester thereof) as an active pharmaceutical ingredient (API), which can be generally referred to herein as a “testosterone API”. Suitable testosterone API's for use in embodiments provided by the present disclosure will preferably be in a stable suspension in a formulation provided by this disclosure (i.e., when combined with the biodegradable polymer and solvent system as described herein). In embodiments, a preferred testosterone API is selected from testosterone or an ester of testosterone. Suitable esters of testosterone for use in embodiments provided by the present disclosure include testosterone undecanoate (TU) which is also known as testosterone undecylate, testosterone cypionate (TC), testosterone propionate, and testosterone busciclate. The testosterone API is in substantially solid form (in suspension) in the biodegradable polymer and solvent(s) composition at temperatures up to body temperature (e.g., about 36.5° C. to about 37.5° C. (about 97.7° F. to about 99.5° F.)). In some embodiments, the testosterone API is in substantially solid form in the extended release formulation at temperatures up to about 35° C., or even at temperatures up to about 40° C. It is desirable for the testosterone API to be chemically and physically stable within the formulation at ambient temperature and at body temperatures, and in some embodiments, at higher temperatures associated with, for example, certain e-beam irradiation processes or other processes which may expose the extended release formulation to an elevated temperature for a period of time. Similarly, because the extended release formulations may be stored for weeks or months at ambient temperatures or under refrigeration, chemical and physical stability of a testosterone API within the extended release formulations in this lower temperature range is also an element of the present disclosure. In embodiments, a testosterone API is in substantially solid form in the extended release formulation at temperatures up to at least about 35° C., or at least about 36° C., or at least about 37° C., or at least about 38° C., or at temperatures up to at least about 39° C., or at least about 40° C. In one embodiment, a testosterone API is in substantially solid form in the extended release composition at a temperature range spanning from refrigeration temperature (e.g., 0-8° C.) or lower up to body temperature, or in other embodiments up to any temperature between 35° C. and 40° C. or higher, in 0.1° C. increments.

As used herein, unless otherwise noted, use of the term “suspension” when referring to a composition of the present disclosure may refer to formulations in which at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90% the testosterone API is in the form of solid particles suspended in the polymer and solvent composition. Description of the testosterone API herein as being “substantially in solid form” or “substantially in suspension” in a formulation refers to formulations in which at least about 50%, or at least about 55%, or at least about 60%, or at least about 65%, or at least about 70%, or at least about 75%, at least about 80%, or at least about 85%, or at least about 90% of the testosterone API is in the form of solid particles suspended in the polymer and solvent composition.

A desired particle size, or distribution of particle sizes, of a testosterone API will largely depend upon the form of testosterone and the desired release profile. In general, as a non-limiting consideration, a smaller particle size will result in more rapid release in vivo (i.e., shorter duration of release) and/or a larger burst and corresponding higher peak concentration in vivo, while a larger particle size will result in slower release in vivo (i.e., longer duration of release) and/or a smaller burst and corresponding lower peak concentration in vivo. In some embodiments, a bimodal particle size distribution may provide an advantageous release profile or other desirable effect; by way of non-limiting example, and without wishing to be bound by any particular theory, it may be possible that smaller particles may cause rapid drug release (e.g., by faster release from a depot and/or faster solubilization upon release and/or modification of fluid channels in the depot) to provide an initial therapeutic effect, and larger particles may be released later to provide an extended therapeutic effect. Embodiments may also comprise particles that have been encapsulated in, for example, a microsphere or lipid sphere, which may provide an additional mechanism for controlling release of testosterone in vivo.

In some embodiments, a testosterone API may be milled to obtain the desired particle size distribution. Suitable milling techniques include, by way of non-limiting example, dry milling, jet milling (also known as fluid energy milling), nanomilling or wet milling in water or other solvent followed by lyophilization or drying, homogenization, ball milling, cutter milling, roller milling, grinding with a mortar and pestle, runner milling, cryomilling, or combinations thereof. In many embodiments, jet milling is a desirable technique due to its temperature control, reduced risk of contamination, and scalability. In general, as a nonlimiting consideration, mechanical micronization and milling techniques are generally more suitable than recrystallization techniques, as recrystallization risks introducing residual solvents and co-crystals that may affect polymer formulation behavior and safety. In some embodiments, a testosterone API may be milled and then lyophilized or otherwise dried to remove residual water and/or improve stability.

As used herein, unless otherwise specified, the term “particle size” refers to a median particle size, also be referred to as “D_{v,50}” values. Additionally, as used herein, unless otherwise specified, the term “span” refers to the difference between a 90th percentile particle size (referred to as “D_{v,90}”) and a 10th percentile particle size (referred to as “D_{v,10}”), divided by the 50th percentile particle size (D_{v,50}); thus, the span of a volume of particles can be interpreted as a measure of how broadly distributed particle sizes are within the volume. The particle sizes (e.g., D_{v,90}, D_{v,50}, and D_{v,10}) are determined by volume-based particle size measurements. Unless otherwise specified, the particle sizes are determined by the use of a laser diffraction particle size analyzer such as a Malvern Mastersizer® instrument. Software programs and calculations that can convert from a number-based distribution analysis to a volume-based distribution analysis (and vice versa) are well known in the art; therefore, for particle sizes calculated using a number-based method, a volume-based particle size can also be estimated. Volume-based particle size distribution measurements are the default choice for many ensemble light scattering particle size measurement techniques, including laser diffraction, and are generally used in the pharmaceutical industry. Further, unless otherwise specified, the particle sizes refer to the size of the API powder, prior to suspension in the composition. Once the API is suspended in the composition, the particle size may be different from that of the raw API powder.

In some embodiments, a testosterone API will have a median particle size (D_{v,50}), prior to suspension in the composition, from about 1 μm to about 100 μm, or from about 1 μm to about 90 μm, or from about 1 μm to about 80 μm, or from about 1 μm to about 70 μm, or from about 1 μm to about 60 μm, or from about 1 μm to about 50 μm, or from about 1 μm to about 40 μm, or from about 1 μm to about 30 μm, or from about 1 μm to about 20 μm. In some embodiments, the median particle size, prior to suspension in the composition, is from about 10 μm to about 100 μm, or from about 20 μm to about 100 μm, or from about 30 μm to about 100 μm, or from about 40 μm to about 100 μm, or from about 50 μm to about 100 μm, or from about 60 μm to about 100 μm, or from about 70 μm to about 100 μm. In some embodiments, the median particle size of a testosterone API, prior to suspension in the composition, is from about 20 μm to about 90 μm, or is from about 25 μm to about 85 μm, or from about 30 μm to about 80 μm, or from about 35 μm to about 75 μm, or from about 40 μm to about 70 μm, or from about 45 μm to about 65 μm. In other embodiments, the median particle size of a testosterone API, prior to suspension in the composition, can range from any whole number to any other whole number from about 1 μm to about 100 μm, both inclusive.

In some embodiments, a testosterone API will have a 90th percentile particle size (D_{v,90}), prior to suspension in the composition, from about 100 μm to about 450 μm, or from about 100 μm to about 440 μm, or from about 100 μm to about 430 μm, or from about 100 μm to about 420 μm, or from about 100 μm to about 410 μm, or from about 100 μm to about 400 μm. In some embodiments, the 90th percentile particle size, prior to suspension in the composition, is from about 200 μm to about 450 μm, or from about 200 μm to about 440 μm, or from about 200 μm to about 430 μm, or from about 200 μm to about 420 μm, or from about 200 μm to about 410 μm, or from about 200 μm to about 400 μm, or from about 300 μm to about 450 μm, or from about 300 μm to about 440 μm, or from about 300 μm to about 430 μm, or from about 300 μm to about 420 μm, or from about 300 μm to about 410 μm, or from about 300 μm to about 400 μm. In other embodiments, a testosterone API will have a 90th percentile particle size (D_{v,90}), prior to suspension in the composition, from about 370 μm to about 450 μm, or from about 380 μm to about 440 μm, or from about 390 μm to about 430 μm, or from about 400 μm to about 420 μm, or from about 300 μm to about 380 μm, or from about 310 μm to about 370 μm, or from about 320 μm to about 360 μm, or from about 330 μm to about 350 μm, or from about 100 μm to about 190 μm, or from about 110 μm to about 180 μm, or from about 120 μm to about 170 μm, or from about 130 μm to about 160 μm. In other embodiments, the 90th percentile particle size of the testosterone API, prior to suspension in the composition, can range from any whole number to any other whole number from about 100 μm to about 450 μm.

In various embodiments, a testosterone API may have, prior to suspension in the composition, a span from about 0.1 to about 9, or from about 0.5 to about 9, or from about 1 to about 9, or from about 1.5 to about 9, or from about 2 to about 9, or from about 2.5 to about 9, or from about 3 to about 9, from about 3.5 to about 9, or from about 4 to about 9, or from about 4.5 to about 9, or from about 5 to about 9. In some embodiments, a testosterone API may have, prior to suspension in the composition, a span from about 1 to about 8.5, or from about 1 to about 8, or from about 1 to about 7.5, or from about 1 to about 7, or from about 1 to about 6.5, from about 1 to about 6, or from about 1 to about 5.5, or from about 1 to about 5, or from about 1 to about 4.5, or from about 1 to about 4, or from about 1 to about 3.5, or from about 1 to about 3, or from about 2 to about 7, or from about 2 to about 6.5, or from about 2 to about 6, or from about 2 to about 5.5, or from about 2 to about 5, or from about 2 to about 4.5, or from about 2 to about 4, or from about 2.5 to about 7, or from about 3 to about 7, or from about 3.5 to about 7, or from about 4 to about 7, or from about 4.5 to about 7, or from about 5 to about 7. In other embodiments, a testosterone API may have, prior to suspension in the composition, a span that can range from any tenth of a whole number to any other tenth of a whole number from about 0.1 to about 9.

In some embodiments, the composition comprises testosterone undecanoate having median particle size (D_{v,50}), prior to suspension in the composition, of between about 35 82 m to about 75 μm, preferably about 45 μm to about 65 μm, and a particle size span of between about 2 to about 7. In other embodiments, the composition comprises testosterone cypionate having median particle size (D_{v,50}), prior to suspension in the composition, of between about 30 μm to about 50 μm and a particle size span of between about 1 to about 3.

The concentration of the testosterone API in the compositions of the present disclosure may range from about 1% to about 40% by weight of the composition, such as from about 1% to about 30% by weight of the composition, or from about 10% to about 30% by weight of the composition, or from about 15% to about 25% by weight of the composition, or from about 18% to about 22% by weight of the composition, or about 20% by weight of the composition. The concentration of the testosterone API in the composition may be about 5% by weight of the composition, or about 10% by weight of the composition, or about 15% by weight of the composition, or about 20% by weight of the composition, or about 25% by weight of the composition, or about 30% by weight of the composition, or about 35% by weight of the composition, or about 40% by weight of the composition. In other embodiments, the amount of testosterone API in the compositions of the present disclosure can range from any whole number percent to any other whole number percent within from about 1% to about 40% by weight of the composition. In some embodiments, the concentration of testosterone API is no more than about 25% by weight of the composition. In some embodiments, the concentration of testosterone API is about 20% by weight of the composition.

According to some embodiments of the present disclosure, the extended release composition may comprise about 100 mg to about 400 mg, or from about 100 mg to about 390 mg, or from about 100 mg to about 380 mg, or from about 100 mg to about 370 mg, or from about 100 mg to about 360 mg, or from about 100 mg to about 350 mg, or from about 100 mg to about 340 mg, or from about 100 mg to about 330 mg, or from about 100 mg to about 320 mg, or from about 100 mg to about 310 mg, or from about 100 mg to about 300 mg, or from about 100 mg to about 290 mg, or from about 100 mg to about 280 mg, or from about 100 mg to about 270 mg, or from about 100 mg to about 260 mg, or from about 100 mg to about 250 mg, or from about 100 mg to about 240 mg, or from about 100 mg to about 230 mg, or from about 100 mg to about 220 mg, or from about 100 mg to about 210 mg, or from about 100 mg to about 200 mg, or from about 150 mg to about 400 mg, or from about 150 mg to about 390 mg, or from about 150 mg to about 380 mg, or from about 150 mg to about 370 mg, or from about 150 mg to about 360 mg, or from about 150 mg to about 350 mg, or from about 150 mg to about 340 mg, or from about 150 mg to about 330 mg, or from about 150 mg to about 320 mg, or from about 150 mg to about 310 mg, or from about 150 mg to about 300 mg, or from about 150 mg to about 290 mg, or from about 150 mg to about 280 mg, or from about 150 mg to about 270 mg, or from about 150 mg to about 260 mg, or from about 150 mg to about 250 mg, or from about 150 mg to about 240 mg, or from about 150 mg to about 230 mg, or from about 150 mg to about 220 mg, or from about 150 mg to about 210 mg, or from about 150 mg to about 200 mg of testosterone API per gram of the composition. In other embodiments, the extended release composition may comprise from any whole number amount to any other whole number amount from about 100 mg to about 400 mg of testosterone API per gram of the composition. In preferred embodiments, the extended release composition comprises from about 150 mg to about 250 mg of testosterone API per gram of the composition.

The amount of testosterone API in the extended release formulation may be sufficient to achieve an average serum testosterone concentration from about 0.5 ng/mL to about 20 ng/mL, or from about 1 ng/mL to about 15 ng/mL, or from about 2 ng/mL to about 15 ng/mL, or from about 3 ng/mL to about 10 ng/mL over the course of one week or longer, or two weeks or longer, or one month or longer, or two months or longer, or three months or longer, or four months or longer, or five months or longer, or six months or longer.

The release profile of the testosterone API from the composition will depend upon several factors, including, but not limited to, the amount, form of testosterone, and particle size distribution, the amount and type of polymer (e.g., monomer ratio, molecular weight, etc.), and the amount and type of solvent/co-solvent. In preferred embodiments, a clinically effective amount of a testosterone API is released in a controlled, or extended, release manner (e.g., with a relatively constant or flat release profile) over the course of at least 3 months, without or with a minimal burst release at shorter release times. The extended release composition, on average during the dosing period, may provide testosterone supplementation in the eugonadal range, wherein the average concentration of testosterone in plasma (C_{avg}) is from about 3 to about 10 ng/mL (i.e., 10.4 nmol/L to 34.7 nmol/L).

The extended release composition of the present disclosure comprises a testosterone API, a solvent system comprising a biocompatible solvent and a low-molecular weight polyethylene glycol (PEG); and a biodegradable polymer comprising co-polymer segments of poly(lactide-co-glycolide) (PLG) and having at least one carboxylic acid end group. In embodiments, the testosterone API may be testosterone undecanoate or testosterone cypionate, preferably with one or more of a D_{v,50} of about 1 μm to about 100 μm, a D_{v,90} of about 100 μm to about 450 μm, and a span of about 1 to about 9, or a span about 2 to about 7. In embodiments, the PLG polymer may have a molar ratio of lactide to glycolide monomers of about 70:30 to about 85:15 and a weight average molecular weight of the biodegradable polymer of about 4 kDa to about 36 kDa. In embodiments, the solvent may comprise N-methyl-2-pyrrolidone (NMP) and one or more PEGs having a number average molecular weight of about 3350 Daltons or less, preferably PEG 250 or PEG 300 or PEG 350 or PEG 400 or combination thereof, and wherein the amount of the low molecular weight PEG is about 25 wt % or less of the composition, or about 15 wt % or less of the composition, or about 10 wt % or less of the composition. In embodiments, the testosterone API makes up about 20 wt % of the composition, the biocompatible solvent system makes up about 50 wt % of the composition, and the biodegradable polymer makes up about 30 wt % of the pharmaceutical composition.

In some embodiments, the extended release composition comprises about 20 wt % of testosterone undecanoate having a D_{v,50} of between about 35 μm to about 75 μm, preferably between about 45 μm to about 65 μm, and a span of between about 2 to about 7, preferably between about 2 to about 4 or between about 5 to about 7; about 50 wt % of a biocompatible solvent system comprising N-methyl-2-pyrrolidone (NMP) and polyethylene glycol having a number average molecular weight of about 300 Daltons (PEG 300), wherein a weight ratio of NMP to PEG 300 is about 4:1; and about 30 wt % of 70:30 poly(D,L-lactide-co-glycolide) (PLG) polymer having at least one carboxylic acid end group and having a weight average molecular weight of between about 4 kDa to about 24 kDa, preferably between about 4 kDa to about 14 kDa. In some embodiments, the weight average molecular weight is about 9 kDa. In some embodiments, the weight average molecular weight is between about 14 kDa to about 24 kDa. In some embodiments, the weight average molecular weight is about 19 kDa.

In other embodiments, the extended release composition, comprises about 20 wt % of testosterone undecanoate having a D_{v,50} of between about 35 μm to about 75 μm, preferably between about 45 μm to about 65 μm, and a span of between about 2 to about 7, preferably between about 2 to about 4 or between about 5 to about 7; about 50 wt % of a biocompatible solvent system comprising N-methyl-2-pyrrolidone (NMP) and polyethylene glycol having a number average molecular weight of about 300 Daltons (PEG 300), wherein a weight ratio of NMP to PEG 300 is about 4:1; and about 30 wt % of 85:15 poly(D,L-lactide-co-glycolide) (PLG) polymer having at least one carboxylic acid end group and having a weight average molecular weight of between about 4 kDa to about 24 kDa, preferably between about 4 kDa to about 14 kDa or between about 14 kDa to about 24 kDa.

Yet in other embodiments, the extended release composition comprises about 20 wt % of testosterone cypionate having a D_{v,50} of between about 30 μm to about 50 μm and a span of between about 1 to about 3; about 50 wt % of a biocompatible solvent system comprising N-methyl-2-pyrrolidone (NMP) and polyethylene glycol having a number average molecular weight of about 300 Daltons (PEG 300), wherein a weight ratio of NMP to PEG 300 is about 3:2; and about 30 wt % of 70:30 poly(D,L-lactide-co-glycolide) (PLG) polymer and having at least one carboxylic acid end group and having a weight average molecular weight of between about 20 kDa to about 36 kDa.

While yet in other embodiments, the extended release composition comprises about 20 wt % of testosterone cypionate having a D_{v,50} of between about 30 μm to about 50 μm and a span of between about 1 to about 3; about 50 wt % of a biocompatible solvent system comprising N-methyl-2-pyrrolidone (NMP) and polyethylene glycol having a number average molecular weight of about 300 Daltons (PEG 300), wherein a weight ratio of NMP to PEG 300 is about 3:2; and about 30 wt % of 85:15 poly(D,L-lactide-co-glycolide) (PLG) polymer and having at least one carboxylic acid end group and having a weight average molecular weight of between about 20 kDa to about 36 kDa.

### Administration

This disclosure also provides methods of testosterone replacement therapy for a condition associated with a deficiency or absence of endogenous testosterone in a patient, comprising administering to the patient the extended release composition disclosed herein. The condition associated with a deficiency or absence of endogenous testosterone may be a congenital or an acquired condition and may be, for example, primary hypogonadism or hypogonadotropic hypogonadism. Alternatively, or additionally the disclosure may provide methods of testosterone supplementation for use as male contraception or in transgender (female-to-male) hormone therapy.

As used herein, the terms “patient” and “subject” are interchangeable and refer generally to an animal (e.g., any organism of the kingdom Animalia including humans and companion animals, such as dogs, cats, and horses; and livestock animals, such as cows, goats, sheep, and pigs) to which a composition or formulation of the present disclosure is administered or is to be administered. In embodiments, the patient is a human. In embodiments, the patient is an adult male. In some embodiments, the adult male patient may have been diagnosed with primary hypogonadism (congenital or an acquired) or hypogonadotropic hypogonadism (congenital or an acquired). In some embodiments, the adult male patient may desire or be in need of male contraception. In some embodiments, the patient may be in need of undergoing, undergoing, or maintaining female to male transition.

In some embodiments, the extended release compositions may be stored at refrigeration or cold storage temperatures from about 0° C. to about 8° C. and then warmed room temperature from about 18° C. to about 25° C. prior to administration to a subject. In some embodiments, the extended release compositions may be stored for a period of 6 months or longer, or 12 months or longer, or 18 months or longer, or 24 months or longer prior to administration to a subject. In some embodiments, the extended release composition may not need to be re-mixed or may be subjected to minimal re-mixing, agitation, shaking, or otherwise disturbing to restore the dosage uniformity prior to being administered to a patient. In some embodiments, the extended release composition may need to be mixed to form the extended release composition or re-mixed prior to administration.

In embodiments, the extended release composition is administered subcutaneously or intramuscularly into the body of a subject. In embodiments, the extended release composition has an injection volume of about 3 mL or less, or preferably about 2 mL or less, or more preferably about 1 mL or less, or even about 0.5 mL. Upon injection of the extended release composition into a subject, the solvent and co-solvent dissipate, and an in-situ solid depot is formed and releases the testosterone API over an extended time period. In various embodiments, the testosterone API within solid depot is released into a patient, in a clinically effective amount (e.g., as determined by measuring average blood serum levels testosterone concentration in a subject).

The extended release composition may be administered weekly, biweekly, monthly, every two months, every three months, every four months, every five months, every six months, and/or for as long as testosterone supplementation is desired. The amount of testosterone API in the extended release formulation may be sufficient to provide one or more initial loading doses at shorter intervals (e.g., weekly, biweekly or monthly), followed by maintenance doses, wherein the amount of testosterone API, provided by the composition and/or the interval of the dosing increases, or under any alternative dosing regimen. The extended release composition may provide testosterone supplementation in the eugonadal range, wherein the average concentration of testosterone in plasma (C_{avg}) is from about 3 ng/mL to about 10 ng/mL (i.e., 10.4 nmol/L to 34.7 nmol/L) during the dosing period. Additionally or alternatively, the extended release composition may be administered at times (i.e., dosing interval) and/or in amounts sufficient to achieve an average serum testosterone concentration from about 0.5 ng/mL to about 20 ng/mL, or from about 1 ng/mL to about 15 ng/mL, or from about 2 ng/mL to about 15 ng/mL, or from about 3 ng/mL to about 10 ng/mL over the course of one week or longer, or two weeks or longer, or one month or longer, or two months or longer, or three months or longer, or four months or longer, or five months or longer, or six months or longer.

### Articles of Manufacture

This disclosure also provides articles of manufacture or kits according to the extended release compositions and administration methods described above. In some embodiments, an article of manufacture of this disclosure includes a container of the extended release composition. The container may be a single syringe, wherein the extended release composition is contained within the syringe. In some embodiments, the syringe may comprise a 16 to 24 gauge needle, preferably a 16 to 22 gauge needle, or more preferably a 16 to 20 gauge needle or an 18 to 20 gauge needle. In other embodiments, the syringe may be an autoinjector syringe.

Another article of manufacture may include a first container comprising the extended release composition described herein, and a second empty container that is used to mix or re-mix the extended release composition prior to administration to a subject. The first and second containers may be first and second chambers of a dual chamber syringe. These articles may also comprise instructions for mixing the composition, comprising connecting the first and second chambers and then pushing the contents of the first chamber into the second chamber and then back into the first chamber, one or more time, until the composition is effectively mixed. These articles may also comprise a 16 to 24 gauge needle, preferably a 16 to 22 gauge needle, or more preferably a 16 to 20 gauge needle or an 18 to 20 gauge needle, which may be attached to the syringe for administration of the extended release composition to the subject.

Another article of manufacture may include a first container comprising the testosterone API and optionally a biocompatible solvent or solvent system, and a second container comprising the biodegradable polymer and the solvent system. In one embodiment, the first container may comprise the testosterone API dry-filled into the first container (i.e., in the absence of any solvent or solvent system), where the second container comprises the biocompatible polymer and the solvent system. In these embodiments, the first and second containers may be first and second chambers of a dual chamber syringe. In these articles, instructions may also be included for mixing the contents of the first and second chambers to form the extended release composition for administration to a subject. The contents of the first and second chambers may be combined within the syringe by adding the contents of second chamber into the first chamber, or vice versa, followed by mixing to form a flowable composition. Alternatively, the contents of the first chamber may be added to the second chamber followed by mixing to form a flowable composition. The first and second containers may also each be a syringe which may be, or are, coupled together to mix the contents together to form a flowable composition. The contents of the first and second chambers may be combined and mixed by coupling the containers together, transferring the contents back and forth between the two chambers until the polymer, solvent system, and the testosterone API are effectively mixed together to form a flowable composition. These articles may also comprise a 16 to 24 gauge needle, preferably a 16 to 22 gauge needle, or more preferably a 16 to 20 gauge needle, or an 18 to 20 gauge needle, which may be attached to the syringe for administration of the extended release composition to the subject.

These articles of manufacture may further comprise instructions for the use thereof for testosterone replacement therapy. These articles of manufacture may also comprise a package insert that provides efficacy and/or safety data for the use of the extended release compositions for testosterone replacement therapy to treat a condition associated with a deficiency or absence of endogenous testosterone.

All publications, patents, and patent documents are incorporated by reference herein, as though individually incorporated by reference.

### EXAMPLES

### Example 1

### The following example describes methods used to prepare and test the extended release compositions comprising testosterone undecanoate (TU) or testosterone cypionate (TC).

PLG Polymers. To produce the formulations described in Examples 2-10 below, acid-initiated poly(D.L-lactide-co-glycolide) copolymers were produced using the following methods. The amounts of DL-lactide, glycolide, and glycolic acid were selected to obtain a targeted monomer molar ratio and weight average molecular weight for each investigated polymer. The monomer molar ratios and weight average molecular weights reported in Examples 2-10 are targeted values; the actual monomer molar ratios and weight average molecular weights may vary slightly due variations in the manufacturing and sterilization processes but are within acceptable specifications and testing limits. In a polymerization vessel, appropriate amounts of DL-lactide, glycolide, and glycolic acid were added, and the vessel contents were placed under a nitrogen atmosphere. The vessel was then lowered into a temperature-controlled oil bath. The temperature of the vessel was increased until the reagents melted. A catalyst solution was made with appropriate amounts of stannous octoate and toluene and added to the vessel. The vessel was then heated to about 135-145° C. under nitrogen atmosphere for about 3-4 hours with constant stirring. Then, to remove unreacted lactide and glycolide monomers, the vessel was evacuated, and the monomers were vacuum distilled out of the polymerization mixture. The hot melt was then extruded into cooling pans. After cooling, the solid mass was broken up into smaller pieces.

TU or TC Polymer/Solvent Formulations. To produce the polymer/solvent formulations comprising the active pharmaceutical ingredient (TU or TC), a PLG polymer of the desired monomer molar ratio and weight average molecular weight were combined with N-Methyl-2-Pyrrolidone (NMP) and PEG, as a solvent and co-solvent, respectively, in the indicated amounts (see individual experiments below). Unless otherwise indicated, the PEG used in these examples is hydroxyl terminated PEG at 300 Da molecular weight. The polymer, NMP, and PEG were combined in a jar blanketed with nitrogen and mixed using a Turbula or jar mill at ambient temperature, or rotisserie at elevated temperature until the composition was homogeneous.

For the testosterone undecanoate formulations used in Examples 2-4 and 6-10 testosterone undecanoate (TU) was processed to have a desired particle size distribution prior to mixing with the copolymer/solvent solution. Specifically, TU particles were either jet milled or Netzch milled. The particle size was determined, prior to adding the TU to the formulation, using volume-based particle size calculation methods using a laser diffraction particle size analyzer, such as Mastersizer® (Malvern Panalytical, Malvern, PA).

To prepare the TU PLG copolymer formulations, testosterone undecanoate was added to the copolymer/solvent solution in amounts that achieve the percentages indicated in Examples below, and manually stirred into the composition until homogenously dispersed. The TU/polymer/solvent mixture was homogenized using a drop down Silverson homogenizer or an IKA Magic Plant homogenizer to allow for injection through a 20 gauge needle. Specifically, when using the drop down Silverson homogenizer, samples were homogenized at 2,500-3,500 rpm for 2-17 minutes. When using the IKA Magic Plant homogenizer, samples were homogenized at 3,000 rpm for 3-6.25 hours. After incorporation of the TU into the polymer/solvent mixture, the formulation was filled into syringes using a semi-automatic syringe filler and the syringes were capped with a luer-luer coupler and male tip cap. Syringes were then packaged in labeled foil pouches with a desiccant pack and the pouches were sealed.

For the testosterone cypionate suspensions used in Examples 5, testosterone cypionate was used as provided by the supplier. To prepare the TC PLG copolymer formulations, testosterone cypionate was added to the copolymer/solvent solution in amounts that achieve the percentages indicated in Example 5, and manually stirred into the composition until homogenously dispersed. The TC PLG copolymer formulations were homogenized using a drop down Silverson homogenizer at 2,000-3,500 rpm for about 5 minutes to allow for injection through a 20 gauge needle. After incorporation of the TC into the polymer/solvent mixture, the formulation was filled into syringes using a semi-automatic syringe filler and the syringes were capped with a luer-luer coupler and male tip cap. Syringes were then packaged in labeled foil pouches with a desiccant pack and the pouches were sealed.

After filling the syringes with the TU- or TC-PLG copolymer formulations, the filled syringes were stored under refrigerated conditions (e.g., 2-8° C.). The syringes were irradiated via with e-beam irradiation. A total irradiation dose of 30 or 32 kGy was administered to reach an approximate total internal dose of 25 kGy. An irradiation scheme of two passes at 15 or 16 kGy with a hold time of at least 1 hour at refrigerated conditions between passes was used to control sample temperature such that it was maintained below the drug dissolution temperature during irradiation. Upon e-beam irradiation, it is noted that the weight average molecular weight of the polymer may be reduce by approximately 0.1-25%, with higher molecular weight polymers typically experiencing a larger reduction within this range than lower molecular weight polymers; therefore, the desired molecular weight of the polymer in the final formulation (post-irradiation) may be different as compared to the initial polymer weight average molecular weight.

Production of Non-Polymeric Testosterone Undecanoate Control Solution. In Example 2, a non-polymeric testosterone undecanoate control solution (also referred to as the “non-polymeric control formulation” was used. To prepare this control formulation, 40 wt. % saline, 40 wt. % PEG 300, and 20 wt. % testosterone undecanoate (TU) were combined and mixed by manual shaking. The formulation was then homogenized using a drop-down Silverson homogenizer at 1,500 rpm for 5 minutes until injectable via a 20 gauge needle. The non-polymeric control solution was manually filled into labeled vials, and vials capped with rubber septa and crimp top lid. Vials were packaged in labeled foil pouches with a desiccant pack and the pouches were sealed. Vials were arranged in a single layer inside a large plastic bag and sent for e-beam irradiation as described above.

In Vivo Release Testing. Testosterone release rates were obtained using a rat model. Castrated male rats were each injected with a single subcutaneous injection of the PLG copolymer formulation comprising 100 mg/kg (0.18 mL), unless otherwise specified, of testosterone undecanoate or 90 mg/kg (0.16 mL) of testosterone cypionate. At predetermined time points, rats were bled and plasma testosterone levels determined. Each data point is based on an average plasma testosterone concentration. Six to ten rats were dosed, with split-dose bleeding performed for early time points. Select rats were sacrificed for depot and histological analysis on days 42, 91, and 147, resulting in an n=3-10 per time point.

Testosterone release rates were also obtained using a minipig model. Castrated male minipigs were injected with at least one but up to seven 1 to 2 mL subcutaneous injections of the PLG copolymer formulation in the neck and inguinal pocket to deliver the indicated dose of testosterone undecanoate. At predetermined time points, minipigs were bled and the plasma testosterone levels determined. Single minipigs were sacrificed for depot and histological analysis on days 42, 91, and 147. Each data point is based on an average plasma testosterone concentration from one to six minipigs.

Testosterone release profiles were obtained by collecting blood samples from the rats or minipigs and then processing the samples to measure the plasma testosterone concentrations by liquid chromatography/mass spectroscopy (LC/MS). Sample collection time points for rats were pre-dose, at 30 minutes, 1 hour, 3 hours, and 10 hours post-dose, and on days 1, 4, 7, 14, 21, 28, 35, 42, 56, 70, 91, 105, 119, 133, and 147 post injection. Sample collection time points for minipigs were pre-dose and on days 1, 7, 14, 21, 28, 35, 42, 56, 70, 91, 105, 119, 133, and 147 post injection. Example 8 also included sample collection at 1, 4, and 8 hours.

In Vitro Release Testing. Testosterone undecanoate release rates were obtained using a USP APPIV in 1× PBS with 3 wt % sodium dodecyl sulfate (SDS). Formulation was delivered into dissolution cells and media recirculated. At predetermined time points, samples were collected and testosterone undecanoate concentration determined by High Performance Liquid Chromatography (HPLC). Sample collection time points for in vitro release were 30 minutes, 3, 5, 8, 15, 24, 36, and 48 hours, then daily for up to 5 days. Cumulative TU release was calculated. Each data point is based on an average of three to six samples.

### Example 2

The following example illustrates the impact of the copolymer monomer molar ratio and molecular weight and the testosterone undecanoate (TU) particle size distribution of a TU-copolymer/solvent formulation on the release characteristics of the formulation in vivo in a rat model.

Several TU-PLG copolymers formulations were prepared according to the methods described in Example 1. All of the formulations used NMP as the solvent and PEG 300 as the co-solvent and the total amount of solvent (i.e., % NMP+% PEG 300) in the formulation was constant at 50 wt. % of the formulation and the weight ratio of NMP to PEG 300 was 4:1. The PLG copolymer was included in an amount of 30 wt. % of the formulation, but varied in its weight average molecular weight and lactide to glycolide monomer molar ratio. Testosterone undecanoate of the indicated particle size was present in all formulations in an amount of 20 wt. % of the formulation. The details of the formulations are provided in Table 1, which lists (1) the composition of each of the formulations with respect to the percentage by weight of: testosterone undecanoate (TU), PLG polymer (PLG), solvent (NMP), and co-solvent (PEG 300); (2) the TU particle size distribution; and (3) the PLG polymer targeted monomer ratio (L:G monomer ratio) and weight average molecular weight (Polymer, MW), post e-beam irradiation of the polymer. Also investigated was a non-polymeric formulation (i.e., a suspension of TU particles in saline and PEG 300), which was prepared according to the procedure outlined Example 1. The details of the non-polymeric formulation are provided in Table 2.

TABLE 1
Composition of TU-acid initiated-PLG Polymer Formulations.

| | | | | | | PLG | PLG |
|---|---|---|---|---|---|---|---|
| Test | Test Formulation | TU Particle Size Distribution | | | | L:G | Polymer |
| Formulation | TU/PLG/NMP/PEG300 | D_{v, 10} | D_{v, 50} | D_{v, 90} | | monomer | MW |
| # | (by weight %) | (μm) | (μm) | (μm) | span | ratio | (kDa)¹ |
| 1 | 20.0/29.7/39.5/9.9 | 1 | 4 | 10 | 2.3 | 70:30 | 9 |
| 2 | 20.0/30.0/40.0/10.0 | 9 | 67 | 412 | 6.0 | 70:30 | 9 |
| 3 | 20.0/30.0/40.0/10.0 | 9 | 67 | 412 | 6.0 | 70:30 | 19 |
| 4 | 20.0/30.0/40.0/10.0 | 6 | 18 | 55 | 2.7 | 70:30 | 19 |
| 5 | 20.0/30.0/40.0/10.0 | 10 | 53 | 340 | 6.2 | 70:30 | 9 |
| 6 | 20.0/30.0/40.0/10.0 | 10 | 53 | 340 | 6.2 | 85:15 | 9 |
| 7 | 20.0/30.0/40.0/10.0 | 10 | 53 | 340 | 6.2 | 70:30 | 19 |

¹Post-e-beam polymer MW indicated.

TABLE 2
Composition of TU Non-Polymeric Control Formulation.

| Test | Test Formulation | TU Particle Size Distribution | | | |
|---|---|---|---|---|---|
| Formulation | TU/PEG300/Saline | D_{v, 10} | D_{v, 50} | D_{v, 90} | |
| # | (by weight %) | (μm) | (μm) | (μm) | Span |
| control | 20.0/40.0/40.0 | 9 | 67 | 412 | 6.0 |

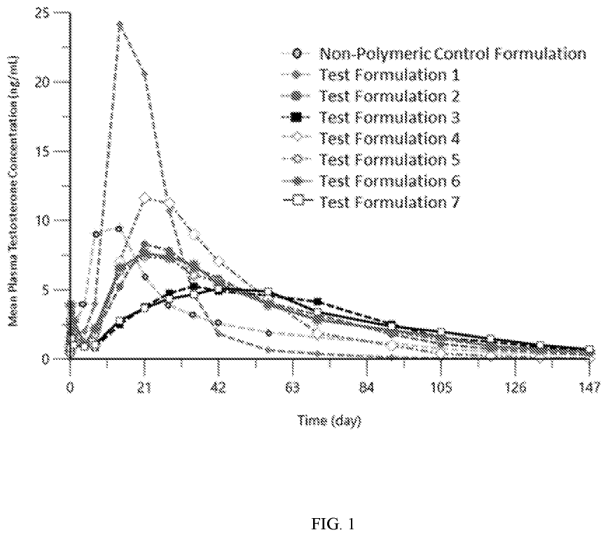

Test Formulations 1-7 and a non-polymeric Control Formulation) were evaluated in vivo in rats according to the method outlined in Example 1. FIG. 1 shows the results of the in vivo release testing (Test Formulations 1 (♦), 2 (●), 3 (▪), 4 (⋄), 5 ( [symbol] ), 6 ( [symbol] ), and 7 ( ) and Control Formulation (○)). The TU release profiles are obtained by measuring the plasma testosterone concentrations before dosing and at various time intervals post dose. Table 3 summarizes the Tₘₐₓ (i.e., the time at which maximum concentration is obtained), Cₘₐₓ (i.e., the maximum concentration), the half-life for each formulation, and the AUC_{inf} (i.e., the area under the curve integrated to obtain the total drug exposure). The Control Formulation (♦) showed somewhat of an extended elevated plasma testosterone levels, with Cₘₐₓ occurring at about 14 days after dosing. Compared to the Control Formulation, all of the Test Formulations with the same or similar TU particle size demonstrated a lower plasma testosterone concentration for longer durations. Only the test formulations with significantly smaller particle size TU showed higher Cₘₐₓ values. In all cases, the observed burst release, (Cₘₐₓ at <2 days), was reasonably low. The variations in the release profile among the Test Formulations are attributed to the differences in the polymer weight average molecular weight and/or the TU particle size distribution.

TABLE 3
PK Parameters for TU-acid initiated-PLG Polymer
and Non-Polymeric Control Formulations.

| Test | | | | AUC_{inf} |
|---|---|---|---|---|
| Formulation | Tₘₐₓ | Cₘₐₓ | Half Life | (days*ng/ |
| # | (days) | (ng/mL) | (days) | mL) |
| 1 | 14 | 24.17 | 12.47 | 491.9 |
| 2 | 21 | 8.29 | 24.08 | 464.1 |
| 3 | 35 | 5.27 | 29.49 | 442.3 |
| 4 | 21 | 11.6 | 16.26 | 483.2 |
| 5 | 21 | 7.4 | 30.9 | 474.9 |
| 6 | 21 | 7.65 | 30.88 | 489.4 |
| 7 | 42 | 5.1 | 34.16 | 444.1 |
| control | 14 | 9.53 | 33.75 | 365.5 |

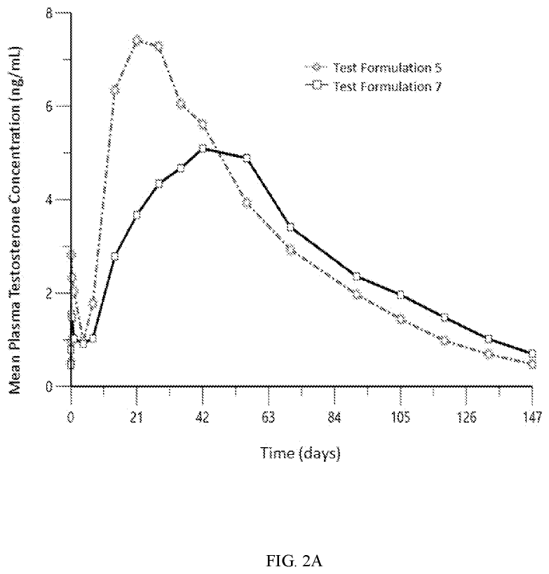

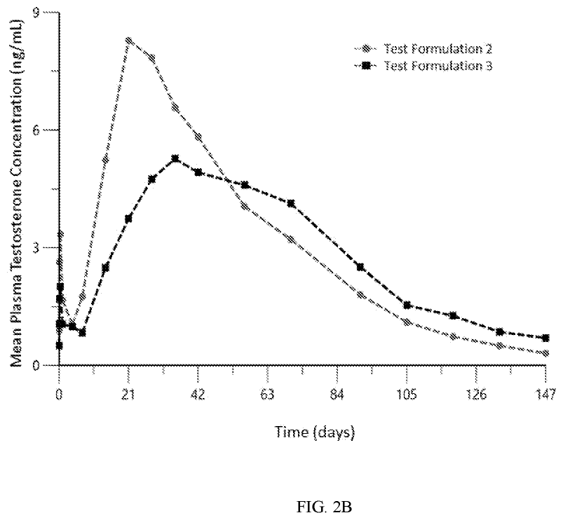

The results of these experiments demonstrate that the weight average molecular weight of the PLG polymer can be used to control the rate and duration of release of TU from the formulation. For ease of comparison, FIGS. 2A and 2B show the impact of varying the PLG polymer weight average molecular weight for otherwise similar formulations. Specifically, FIG. 2A shows the TU release profiles of Test Formulations 5 ( [symbol] ) and 7 ( ), which have the same lactide to glycolide monomer ratio and TU particle size distribution but differ in that Test Formulation 5 has a weight average molecular weight of 9 kDa and Test Formulation 7 has a weight average molecular weights of 19 kDa. FIG. 2B shows the TU release profile of Test Formulations 2 (●) and 3 (▪), which have the same lactide to glycolide monomer ratio and TU particle size distribution but differ in that Test Formulation 2 has a weight average molecular weight of 9 kDa and Test Formulation 3 has a weight average molecular weights of 19 kDa. The two test formulations shown in FIG. 2B have a larger particle size (D_{v,50} and Dᵥ₉₀) compared to the two test formulations shown in FIG. 2A. The results show that by increasing the weight average molecular weight of the PLG polymer in the formulation, the Tₘₐₓ can be extended, and the Cₘₐₓ can be decreased. As the molecular weight of the polymer increases, the release curve generally tends to flatten, lowering the Cₘₐₓ and extending the duration of elevated plasma T levels. At very early times the initial (burst) release also appears to be less for the formulations with the higher weight average molecular weight.

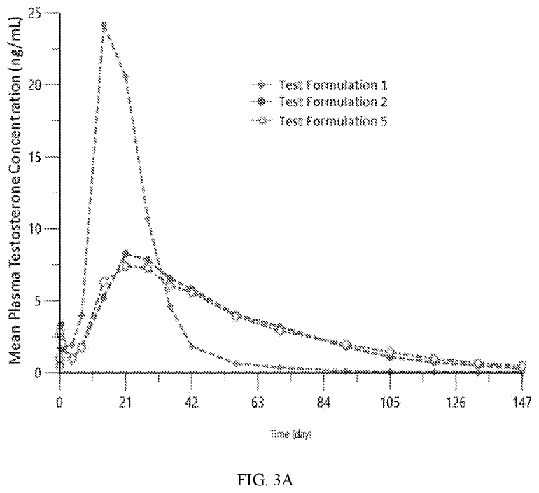

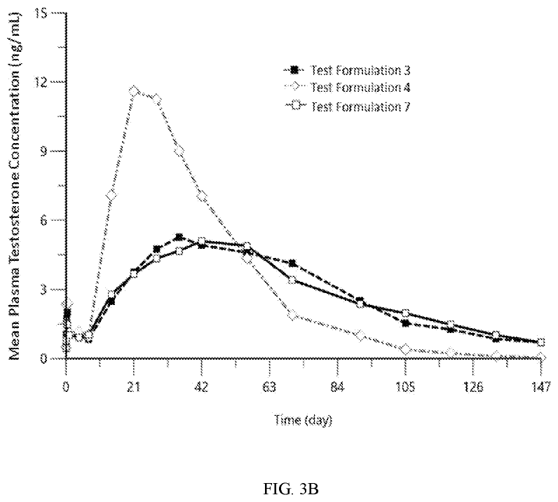

The experimental results also show that the TU particle size distribution can be used to control the rate and duration of release of TU from the formulation. FIGS. 3A and 3B shows the impact of varying the TU particle size distribution. Specifically, FIG. 3A shows the TU release profiles of Test Formulations 1 (♦), 2 (●), and 5 ( [symbol] ), which have the same lactide to glycolide monomer ratio and a weight average molecular weight of 9 kDa but differ in that Test Formulation 1 has a D_{v,50} of 4 and span of 2.3, Test Formulation 5 has a D_{v,50} of 53 and span of 6.2, and Test Formulation 2 has a D_{v,50} of 67 and span of 6. FIG. 3B shows the TU release profile of Test Formulations 3 (▪), 4 (⋄), and 7 ( ), which have the same lactide to glycolide monomer ratio and a similar weight average molecular weight of 19 kDa but differ in that Test Formulation 4 has a D_{v,50}, of 18 and span of 2.7, Test Formulation 7 has a D_{v,50}, of 53 and span of 6.2, and Test Formulation 3 has a D_{v,50}, of 67 and span of 6. These results show that formulations comprising TU particles with a lower D_{v,50} result in a shorter duration and a higher Cₘₐₓ, when compared to formulations comprising TU particle that are larger in size. The effect of the particle size distribution on plasma testosterone profile was more pronounced in the lower molecular weight formulations (e.g., the 9 kDa formulations), where among the formulations, Test Formulation 1 has the smallest TU particle size of 4 μm and has a much shorter duration compared to Test Formulations 2 and 5. In contrast, for the higher molecular weight formulations (e.g., the 19 kDa formulations), Test Formulation 4 has a TU particle size of 18 μm and, while it displays a shorter duration and a higher Cₘₐₓ compared to Test Formulations 3 and 7, the difference in the plasma testosterone profile is less pronounced even though it has a much smaller particle size compared Test Formulations 3 and 7. Interestingly, the particle size appeared to have little or no impact on the initial (burst) release, at least under these conditions.

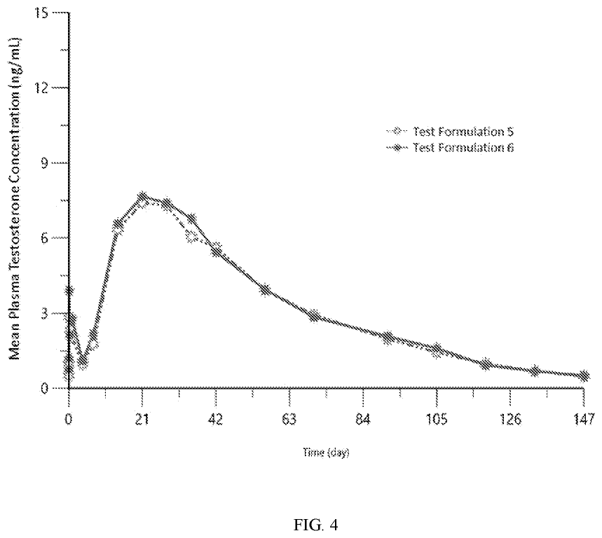

FIG. 4 shows the plasma testosterone profiles for Test Formulations 5 (⋄) and 6 (♦). Test Formulations 5 and 6 both contain TU having the same particle size distribution and they are both a 9 kDa PLG copolymer, but the polymer differs in the lactide to glycolide monomer molar ratio, which for Test Formulation 5 is 70:30, while for Test Formulation 6 it is 85:15. FIG. 4 shows that despite the difference in lactide to glycolide monomer ratio, the two plasma testosterone profiles for Test Formulations 5 and 6 are similar. Surprisingly, and contrary to expectations, the lactide to glycolide monomer ratio seemed to have no substantial impact on the TU release profile under these conditions (e.g., lower weight average molecular weight).

Taken together, the release profile of the PLG copolymer formulations is impacted by the weight average molecular weight of the polymer and the TU particle size distribution. These variables can be used to tune the TU release profile to obtain optimal release kinetics.

### Example 3

The following example illustrates the impact the dose proportionality of a TU-copolymer/solvent formulation on the plasma testosterone profile and PK characteristics of the formulation in vivo in a rat model.

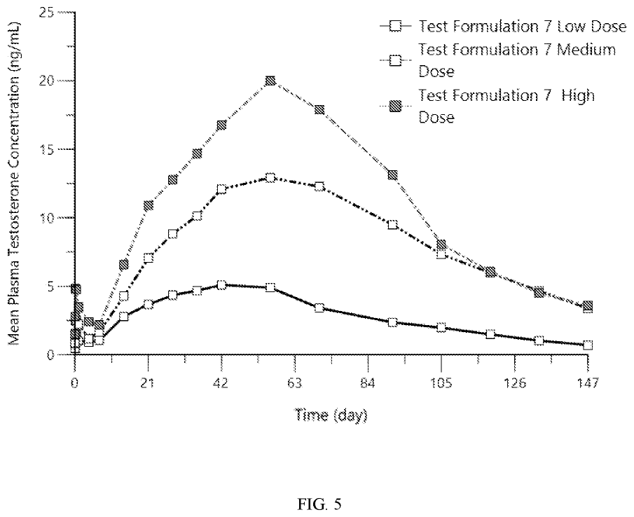

Test Formulation 7 from Example 2 was injected into rats at different doses. Test Formulation 7 is a 30 wt. % acid initiated-poly(D,L-lactide-co-glycolide) copolymer with a targeted L:G monomer ratio of 70:30 and weight average molecular weight of 19 kDa, 40 wt. % NPM, 10 wt. % PEG 300, and 20 wt. % TU having a D_{v,50} of 53 μm and a span of 6. FIG. 5 shows the TU in vivo release profile for rats receiving a low dose of 100 mg/kg (0.18 mL) (--), a medium dose of 300 mg/kg (0.53 mL) ( [symbol] ), and a high dose of 500 mg/kg (0.88 mL) ( [symbol] ). The Tₘₐₓ, Cₘₐₓ, half-life, AUC_{inf} and AUC_{inf/dose} are provided in Table 4. The results show that increasing the dosage amount increases the Cₘₐₓ and AUC_{inf}, while having minimal effect on the Tₘₐₓ and half-life.

TABLE 4
PK Parameters for TU-acid initiated-PLG Polymer
Formulations at Increasing Doses in Rats

| Test | Formu- | | | | | AUC_{INF}/ |
|---|---|---|---|---|---|---|
| Formu- | lation | | | Half | AUC_{inf} | Dose |
| lation | Dose (mg | Tₘₐₓ | Cₘₐₓ | Life | (days*ng/ | (days*ng/ |
| # | TU/kg) | (days) | (ng/mL) | (days) | mL) | mL/mg) |
| 7 | 100 | 42 | 5.1 | 34.16 | 444.07 | 23.03 |
| | 300 | 56 | 12.9 | 41.61 | 1395.21 | 22.54 |
| | 500 | 56 | 22.37 | 34.94 | 2066.58 | 20.32 |

### Example 4

The results from Example 2 show that the TU particle size distribution can be used to control the rate and duration of release of TU from the formulation. Specifically, formulations comprising TU particles having a larger size had a flatter profile with a longer duration of TU release. In this example, the impact the testosterone undecanoate (TU) particle size distribution, specifically the D_{v,90} and the span of the distribution, of a TU-copolymer/solvent formulation on the release characteristics of the formulation in vivo in a rat model is investigated.

Test Formulation 8 was prepared according to the methods described in Example 1. The composition of Test Formulation 8 is summarized in Table 5.

TABLE 5
Composition of TU-acid initiated-PLG Polymer Formulations.

| | | | | | | PLG | PLG |
|---|---|---|---|---|---|---|---|
| Test | Test Formulation | TU Particle Size Distribution | | | | L:G | Polymer |
| Formulation | TU/PLG/NMP/PEG300 | D_{v, 10} | D_{v, 50} | D_{v, 90} | | monomer | MW |
| # | (by weight %) | (μm) | (μm) | (μm) | Span | ratio | (kDa)¹ |
| 8 | 20.0/30.0/40.0/10.0 | 11 | 51 | 146 | 2.6 | 70:30 | 19 |

¹Post e-beam polymer MW indicated.

Test Formulation 8 is similar in composition to Test Formulations 3 and 7 of Example 2. Test Formulation 3 comprises TU particles having a D_{v,50} of 67 μm, a D_{v,90} of 412 μm, and a span of 6 and Test Formulation 7 comprises TU particles having a D_{v,50} of 53 μm, a D_{v,90} of 340 μm, and a span of 6.2, but otherwise the formulations are similar to Test Formulation 8. All three formulations have comparable D_{v,50}; the primary difference is that Test Formulation 8 has a significantly smaller D_{v,90} and span.

TABLE 6
PK Parameters for TU-acid initiated-PLG Polymer
Formulations with Differing TU PSD

| Test | | | | AUC_{inf} |
|---|---|---|---|---|
| Formulation | Tₘₐₓ | Cₘₐₓ | Half Life | (days*ng/ |
| # | (days) | (ng/mL) | (days) | mL) |
| 3 | 35 | 5.27 | 29.49 | 442.3 |
| 7 | 42 | 5.1 | 34.16 | 444.1 |
| 8 | 56 | 7.56 | 23.95 | 536.9 |

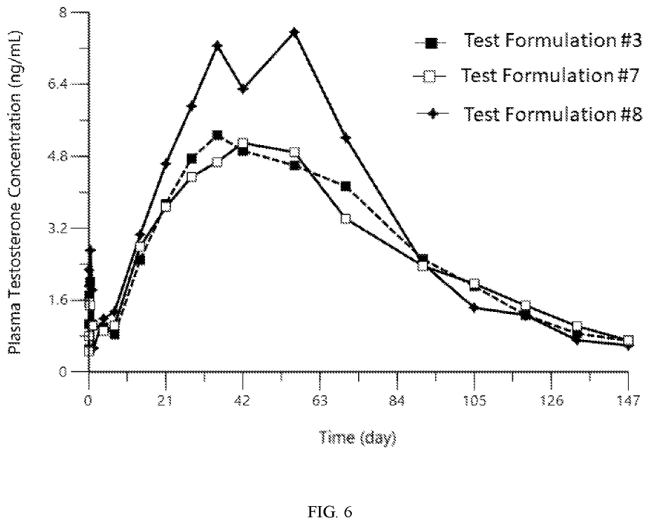

FIG. 6 shows the TU release profile of Test Formulation 8 (♦), along with the release profiles of Test Formulations 3 (▪) and 7 ( ) from Example 2, obtained in vivo using the rat model. Table 6 summarizes the Tₘₐₓ, Cₘₐₓ, half-life, AUC_{inf} for each formulation. All three formulations provide a similar duration of release, but Test Formulation 8 appears to have a bimodal profile with a greater Cₘₐₓ, later Tₘₐₓ, and steeper elimination phase. The initial burst release (Cₘₐₓ at <2 days) was low for all three formulations. These results show that, in addition to the D_{v,50}, the D_{v,90} and D_{v,10} (and thus the span) can be used to tune the TU release profile.

### Example 5

The following example illustrates the impact of the copolymer monomer ratio and molecular weight and solvent composition of a TC-PLG copolymer formulation on the release characteristics of the formulation in vivo in a rat model.

Several TC-PLG copolymers formulations were prepared according to the methods described in Example 1. All of the formulations used NMP as the solvent and PEG 300 as the co-solvent and the total amount of solvent (i.e., % NMP+% PEG 300) in the formulation was constant at 50 wt. % of the formulation, but the weight ratio of NMP to PEG 300 was varied. The PLG copolymer was included in an amount of 30 wt. % of the formulation but varied in its weight average molecular weight and lactide to glycolide (L:G) monomer molar ratio. Testosterone cypionate was used as provided by the supplier and included in the formulations in an amount of 20 wt. % of the formulation. The details of the formulations are provided in Table 7, which lists (1) the composition of each of the formulations with respect to the percentage by weight of: testosterone cypionate (TC), PLG polymer (PLG), solvent (NMP), and co-solvent (PEG 300); (2) the TC particle size distribution; and (3) the PLG polymer targeted monomer ratio (L:G monomer ratio) and weight average molecular weight (Polymer, MW), post e-beam irradiation of the polymer.

TABLE 7
Composition of TC-acid initiated-PLG Polymer Formulations.

| | | | | | | PLG | PLG |
|---|---|---|---|---|---|---|---|
| Test | Test Formulation | TC Particle Size Distribution | | | | L:G | Polymer |
| Formulation | TC/PLG/NMP/PEG300 | D_{v, 10} | D_{v, 50} | D_{v, 90} | | monomer | MW |
| # | (by weight %) | (μm) | (μm) | (μm) | Span | ratio | (kDa)¹ |
| 9 | 20.0/30.0/40.0/10.0 | 16 | 41 | 85 | 1.7 | 70:30 | 19 |
| 10 | 20.0/30.0/30.0/20.0 | 18 | 38 | 72 | 1.4 | 70:30 | 24 |
| 11 | 20.0/30.0/30.0/20.0 | 18 | 38 | 72 | 1.4 | 85:15 | 28 |

¹Post e-beam polymer MW indicated.

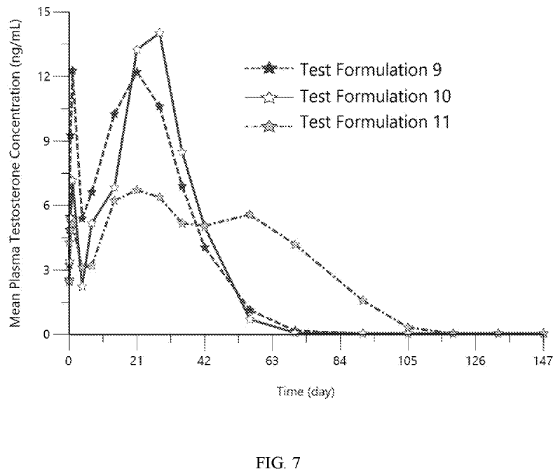

Test Formulations 9-11 were evaluated in vivo in rats according to the method outlined in Example 1. FIG. 7 shows the results of the in vivo release testing (Test Formulations 9 ( [symbol] ), 10 ( [symbol] ), and 11 ( [symbol] )). The TC release profiles are obtained by measuring the plasma testosterone concentrations before dosing and at various time intervals post dose. All of the Test Formulations demonstrated the ability to provide extended duration of elevated plasma T levels. Table 8 summarizes the Tₘₐₓ, Cₘₐₓ, half-life, and AUC_{inf} for each formulation. Of the TC formulations, Test Formulation 11 showed the most favorable plasma testosterone profile, having a longer half-life and a lower Cₘₐₓ when compared to the other two formulations. Notably, Test Formulation 11 has a greater amount of lactide monomers versus glycolide monomers (e.g., 85:15 versus 70:30). This result is in contrast to the results shown in Example 2 for Test Formulations 4 and 5, which showed little impact of the lactide to glycolide monomer ratio (See FIG. 4). The amount of PEG 300 included in these TC formulations is also higher. It is also interesting that the release profile of Test Formulation 11, following the initial burst release, appears to be bimodal with Cₘₐₓ occurring at about 21 days and then a second lesser maximum at about 56 days. The observed burst release, (Cₘₐₓ at <2 days), was reasonably low for each formulation.

TABLE 8
PK Parameters for TC-acid initiated-PLG Polymer Formulations

| | | | | AUC_{inf} |
|---|---|---|---|---|
| Test Formulation | Tₘₐₓ | Cₘₐₓ | Half Life | (days*ng/ |
| # | (days) | (ng/mL) | (days) | mL) |
| 9 | 21 | 12.21 | 7.54 | 421.8 |
| 10 | 28 | 14.02 | 6.46 | 414.8 |
| 11 | 21 | 6.7 | 15.26 | 458.7 |

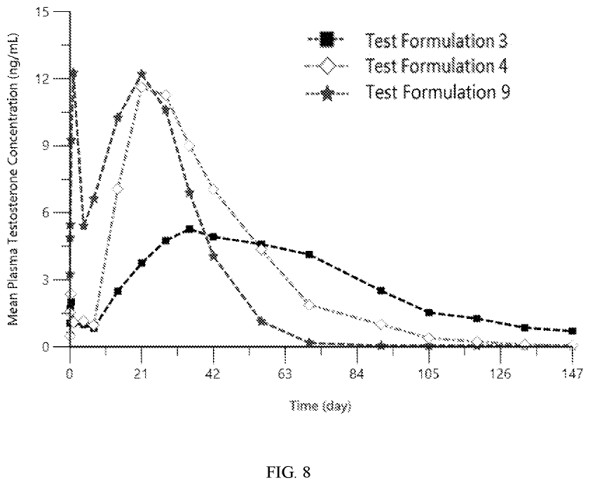

The plasma testosterone profile obtained with TC-PLG formulations with the same NMP:PEG ratio and polymer has a higher initial burst release when compared to the TU-PLG copolymer formulation in Example 2 (formulation 3). Further, for a given polymer formulation (e.g., weight average molecular weight and monomer ratio), the duration of release is shorter. FIG. 8 shows a comparison of the release profile of Test Formulation 9 ( [symbol] ) with that of TU-Test Formulations 3 (▪) and 4 (⋄) in Example 2. All three formulations utilize a 19 kDa 70:30 poly(D.L-lactide-glycolide) copolymers. The TC particle size (D_{v,50}) is 41 μm, which is between the TU particle sizes in Test Formulations 3 and 4 in Example 2 of 67 μm and 18 μm, respectively. The release profile of Test Formulation 9 is shorter in duration, even compared to that of TU-Test Formulations 3, which has a much smaller particle size of the active ingredient. Without being limited to any particular theory, it is possible that the increase in release rate in TC form with larger particle size (41 um) than otherwise similar TU formulations is due to the difference in solubility between the two esters.

The results of these experiments demonstrate that the PLG polymer formulation can be tuned to provide extended release of TC. Of the TC formulations, Test Formulation 11 showed the most favorable plasma testosterone profile based on and the lower Cₘₐₓ values and higher half-life values. Under these conditions, the lactide to glycolide monomer ratio can be used to tune the plasma testosterone profile to obtain optimal release kinetics.

### Example 6

The following example illustrates the impact of polymer molecular weight of a TU PLG copolymer formulation on the release characteristics of the formulation in vivo in a mini pig animal model.

Two TU-poly(D,L-lactide-glycolide) copolymers formulations were prepared according to the methods described in Example 1. The two formulations differ from each other in the weight average molecular weight of the PLG copolymer. The details of the formulations are provided in Table 9, which lists (1) the composition of each of the formulations with respect to the percentage by weight of: testosterone undecanoate (TU), PLG polymer (PLG), solvent (NMP), and co-solvent (PEG 300); (2) the TU particle size distribution; and (3) the PLG polymer targeted monomer ratio (L:G monomer ratio) and weight average molecular weight (Polymer, MW), post e-beam irradiation of the polymer. Test Formulation 13 is the same as that of Test Formulation 8 in Example 4.

TABLE 9
Composition of TU-acid initiated-PLG Polymer Formulations.

| | | | | | | PLG | PLG |
|---|---|---|---|---|---|---|---|
| Test | Test Formulation | TU Particle Size Distribution | | | | L:G | Polymer |
| Formulation | TU/PLG/NMP/PEG300 | D_{v, 10} | D_{v, 50} | D_{v, 90} | | monomer | MW |
| # | (by weight %) | (μm) | (μm) | (μm) | Span | ratio | (kDa) |
| 12 | 20.0/30.0/40.0/10.0 | 11 | 51 | 146 | 2.6 | 70:30 | 9 |
| 13 | 20.0/30.0/40.0/10.0 | 11 | 51 | 146 | 2.6 | 70:30 | 19 |

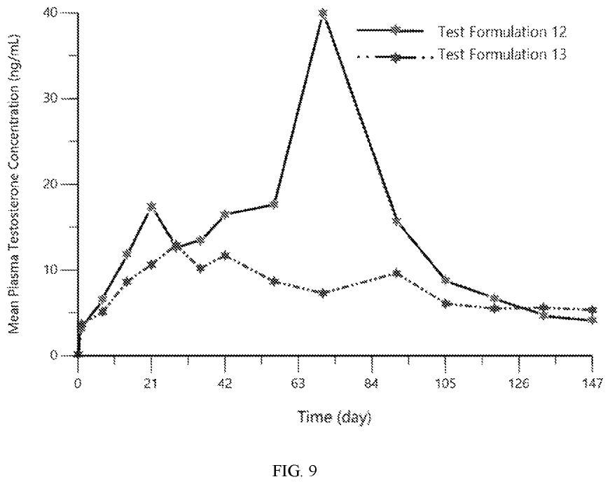

Test Formulations 12 and 13, were injected into minipigs and the plasma testosterone profiles are obtained by measuring the in plasma concentrations before dosing and at various time intervals post dose as described in Example 1. FIG. 9 shows the results of the in vivo release testing (Test Formulations 12 ( [symbol] ) and 13 ( [symbol] )). Table 10 summarizes the PK parameters Tₘₐₓ, Cₘₐₓ, half-life, and AUC_{inf} for the two formulations. Test Formulations 12 and 13 confirmed the ability of the PLG copolymer formulation to provide extended release of TU in the minipig model, with elevated plasma T levels observed for 5 months. The TU-PLG copolymer formulation having the 19 kDa copolymer (Test Formulation 13) appears to produce higher plasma T levels with a later Tₘₐₓ and higher Cₘₐₓ than that of the 9 kDa copolymer (Test Formulation 12). The sharp Cₘₐₓ observed in the release profile for Test Formulation 13 may be due to the small sample size, as an n=1-3 was used for various time points in the minipigs study.

TABLE 10
PK Parameters for TU-acid initiated-
PLG Polymer Formulations in Minipigs

| | | | | | AUC_{inf} |
|---|---|---|---|---|---|
| Test Formulation | TU Dose | Tmax | Cmax | Half Life | (days*ng/ |
| # | (mg/kg) | (days) | (ng/mL) | (days) | mL) |
| 12 | 300 | 25.44 | 12.93 | 237.68 | NC |
| 13 | 300 | 39.52 | 26.26 | 60.55 | 1913.0 |

NC: AUCinf was not calculated

### Example 7

To investigate the effect of lactide-glycolide monomer ratio in TU-PLG copolymer formulations having a weight average molecular weight of about 19 kDa (post-e-beam), additional TU-PLG copolymer formulations are prepared according to the methods described in Example 1. In this example, all of the formulations comprise: (a) a co-solvent system having NMP as solvent and PEG 300 as co-solvent, where the total amount of solvent (i.e., % NMP+% PEG 300) in the formulation is 50 wt. % of the formulation and the weight ratio of NMP to PEG 300 is 4:1; (b) an acid-initiated poly(D,L-lactide-co-glycolide) (PLG) copolymer having a post-e-beam weight average molecular weight of about 19 kDa, at an amount of 30 wt. % of the formulation, where some formulations have a target lactide-glycolide monomer ratio of 70:30 and other formulations have a target lactide-glycolide monomer ratio of 85:15; and (c) testosterone undecanoate (TU) in an amount of 20 wt. % of the formulation, having a Dv,50 of between about 45 μm and about 75 μm (or targeting about 53 μm or about 67 μm) and a Dv,90 of between about 300 μm and about 450 μm (or targeting about 340 μm or about 412 μm). For Example, Test Formulations 3 and 7 are examples of Test Formulations having a target lactide-glycolide monomer ratio of 70:30 (see Example 2). Similar Test Formulations are prepared but using a target lactide-glycolide monomer ratio of 85:15.

These additional Test Formulations are evaluated in vivo in rats according to the method outlined in Example 1. The TU release profiles are obtained by measuring the plasma testosterone concentrations before dosing and at various time intervals post dose as described in the Examples above. The results of this experiment demonstrate the impact of polymer monomer ratio in a 19 kDa polymer formulation on the control of the rate and duration of release of TU from the formulation.

### Example 8

The following example illustrates the impact the dose proportionality of a TU-copolymer/solvent formulation on the plasma testosterone profile and PK characteristics of the formulation in vivo in a minipig model for a TU-PLG copolymer formulation having a weight average molecular weight of about 19 kDa (post-e-beam).

TU-PLG copolymer formulation was prepared according to the methods described in Example 1. The details of the formulation are given in Table 11

TABLE 11
Composition of TU-acid initiated-PLG Polymer Formulations.

| | | | | | | PLG | PLG |
|---|---|---|---|---|---|---|---|
| Test | Test Formulation | TU Particle Size Distribution | | | | L:G | Polymer |
| Formulation | TU/PLG/NMP/PEG300 | D_{v, 10} | D_{v, 50} | D_{v, 90} | | monomer | MW |
| # | (by weight %) | (μm) | (μm) | (μm) | Span | ratio | (kDa) |
| 14 | 20.0/30.0/40.0/10.0 | 12 | 58 | 258 | 4.2 | 70:30 | 19 |

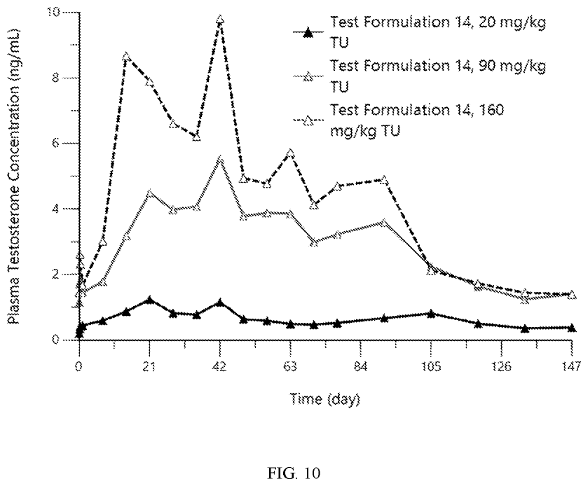

Test Formulation 14 was injected into minipigs at different doses. Test Formulation 14 is a 30 wt. % acid initiated-poly(D,L-lactide-co-glycolide) copolymer with a targeted L:G monomer ratio of 70:30 and weight average molecular weight of 19 kDa, 40 wt. % NMP, 10 wt. % PEG 300, and 20 wt. % TU having a D_{v,50} of 58 μm and a span of 4. FIG. 10 shows the TU in vivo plasma testosterone profile for minipigs receiving a low dose of 20 mg/kg (1× 1 mL) (black -▴- ), a medium dose of 90 mg/kg (3× 1.5 mL) (grey [symbol] ), and a high dose of 160 mg/kg (4× 2 mL) (dashed [symbol] ). The Tₘₐₓ, Cₘₐₓ, half-life, and AUC_{inf}, and AUC_{inf/dose} are provided in Table 12. The results show that increasing the dosage amount increases the Cₘₐₓ, while having minimal effect on the Tₘₐₓ.

TABLE 12
PK Parameters for TU-acid initiated-PLG Polymer
Formulations at Increasing Doses in Minpigs

| Test | Formu- | | | | | AUC_{INF}/ |
|---|---|---|---|---|---|---|
| Formu- | lation | | | Half | AUC_{inf} | Dose |
| lation | Dose (mg | Tₘₐₓ | Cₘₐₓ | Life | (days*ng/ | (days*ng/ |
| # | TU/kg) | (days) | (ng/mL) | (days) | mL) | mL/mg) |
| 14 | 20 | 34.42 | 1.18 | 25.08 | 70.33 | 0.46 |
| | 90 | 36.15 | 6.12 | 51.10 | N/C | N/C |
| | 160 | 30.86 | 11.00 | 47.65 | N/C | N/C |

N/C: Not calculated due to high AUC % Extrapolation values.

### Example 9

To investigate the effect of dose proportionality of a TU-copolymer/solvent formulation on the plasma testosterone profile and PK characteristics of the formulation in vivo in a minipig model, TU-PLG copolymer formulations having a weight average molecular weight of about 9 kDa (post-e-beam) are prepared according to the methods described in Example 1.

In this example, the formulation comprises: (a) a co-solvent system having NMP as solvent and PEG 300 as co-solvent, where the total amount of solvent (i.e., % NMP+% PEG 300) in the formulation is 50 wt. % of the formulation and the weight ratio of NMP to PEG 300 is 4:1; (b) an acid-initiated poly(D,L-lactide-co-glycolide) (PLG) copolymer having a post-e-beam weight average molecular weight of about 9 kDa and a target lactide-glycolide monomer ratio of 70:30, at an amount of 30 wt. % of the formulation; and 20 wt. % TU having a D_{v,50} of 45 to 75 μm and a span of about 4 to 6.

This additional Test Formulation is evaluated in vivo in minipigs according to the method outlined in Example 1, at various doses similar to as described in Example 8. The plasma testosterone profiles are obtained by measuring the plasma testosterone concentrations before dosing and at various time intervals post dose as described in the Examples above. The results of this experiment demonstrate the impact of dose proportionality in a 9 kDa polymer formulation on the plasma testosterone profile and PK parameters.

### Example 10

To investigate the effect of PEG molecular weight and end group on the in vitro release characteristics of the TU-copolymer/solvent formulation, additional TU-PLG copolymer formulations were prepared according to the methods described in Example 1.

TABLE 13
Composition of TU-acid initiated-PLG Polymer Formulations.

| | | | | | | | PLG | PLG |
|---|---|---|---|---|---|---|---|---|
| Test | Test Formulation | | TU Particle Size Distribution | | | | L:G | Polymer |
| Formulation | TU/PLG/NMP/PEG | | D_{v, 10} | D_{v, 50} | D_{v, 90} | | monomer | MW |
| # | (by weight %) | PEG Used | (μm) | (μm) | (μm) | Span | ratio | (kDa) |
| 15 | 20.0/30.0/40.0/10.1 | PEG, 300 Da | 11 | 48 | 242 | 4.8 | 70:30 | 19 |
| 16 | 20.0/30.0/39.9/10.0 | PEG mono- | 11 | 48 | 242 | 4.8 | 70:30 | 19 |
| | | methyl ether, | | | | | | |
| | | 350 Da | | | | | | |
| 17 | 20.0/30.0/40.0/10.0 | PEG | 11 | 48 | 242 | 4.8 | 70:30 | 19 |
| | | dimethyl | | | | | | |
| | | ether, 250 | | | | | | |
| | | Da | | | | | | |

Note:
Composition may not add up to 100% due to sig figs/rounding.

In this example, the formulation comprises: (a) a co-solvent system having NMP as solvent and PEG as co-solvent, where the total amount of solvent (i.e., % NMP+% PEG) in the formulation is 50 wt. % of the formulation and the weight ratio of NMP to PEG is 4:1; (b) an acid-initiated poly(D,L-lactide-co-glycolide) (PLG) copolymer having a post-e-beam weight average molecular weight of about 19 kDa and a target lactide-glycolide monomer ratio of 70:30, at an amount of 30 wt. % of the formulation; and 20 wt. % TU having a D_{v,50} of 48 μm and a span of 5.

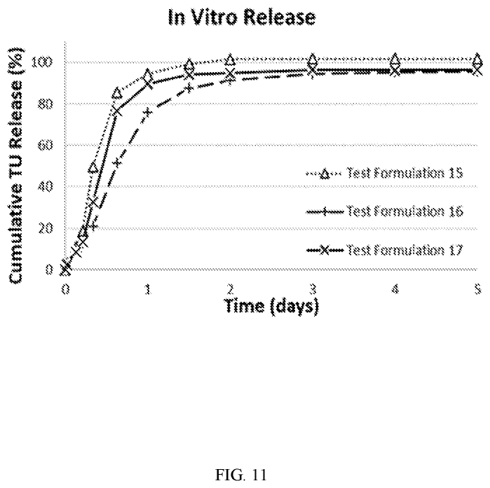

These additional Test Formulations 15, 16, and 17 were evaluated in vitro according to the method outlined in Example 1. FIG. 11 shows the results of the in vivo release testing (Test Formulations 15 ( [symbol] ), 16 (--+--), and 17 (-X-), shown as the cumulative TU release. The results of this experiment demonstrate that various low molecular weight PEGs with a variety of end groups and molecular weights can be used in the formulation while maintaining desirable rate and duration of TU release from the formulation.

These additional Test Formulations (15, 16, and 17) are evaluated in vivo in rats according to the method outlined in Example 1. The TU release profiles are obtained by measuring the plasma testosterone concentrations before dosing and at various time intervals post dose as described in Example 1 above. The results of this experiment demonstrate the impact of PEG end ground and PEG molecular weight on the control of plasma testosterone profile and PK parameters achieved using the TU-PLG formulations.

Various modifications of the embodiments described herein will be evident to those skilled in the art. It is intended that such modifications are included within the scope of the following claims.

1. A pharmaceutical extended release composition, comprising:
an active pharmaceutical ingredient comprising testosterone or a pharmaceutically acceptable ester thereof, wherein the active pharmaceutical ingredient, prior to suspension in the pharmaceutical composition, has a D_{v,50} of about 1 μm-about 100 μm;
a solvent system comprising a biocompatible solvent and a low-molecular weight polyethylene glycol (PEG) having a number average molecular weight of about 3350 Daltons or less; and
a biodegradable polymer comprising co-polymer segments of poly(lactide-co-glycolide) (PLG) having a molar ratio of lactide to glycolide monomers of about 50:50 to about 90:10, at least one carboxylic acid end group, and a weight average molecular weight of about 1 kDa-about 45 kDa.

2. The pharmaceutical extended release composition of claim 1, wherein the active pharmaceutical ingredient is selected from the group consisting of testosterone undecanoate and testosterone cypionate.

3. (canceled)

4. The pharmaceutical composition of claim 2, wherein an amount of testosterone undecanoate or testosterone cypionate in the composition is from about 100 mg-to about 400 mg per gram of the pharmaceutical composition.

5. (canceled)

6. (canceled)

7. (canceled)

8. (canceled)

9. (canceled)

10. (canceled)

11. The pharmaceutical extended release composition of claim 1, wherein the active pharmaceutical ingredient, prior to suspension in the pharmaceutical composition, has a D_{v,50} of about 35 μm-about 75 μm.

12. The pharmaceutical extended release composition of claim 1, wherein the active pharmaceutical ingredient, prior to suspension in the pharmaceutical composition, has a D_{v,90} of about 100 μm-about 450 μm.

13. (canceled)

14. The pharmaceutical extended release composition of claim 1, wherein the active pharmaceutical ingredient, prior to suspension in the pharmaceutical composition, has a span of about 1-about 9.

15. (canceled)

16. (canceled)

17. (canceled)

18. (canceled)

19. The pharmaceutical extended release composition of claim 1, wherein the amount of the low molecular weight PEG is about 25 wt. % or less, about 15 wt. % or less or about 10 wt. % or less of the pharmaceutical composition.

20. (canceled)

21. (canceled)

22. (canceled)

23. (canceled)

24. The pharmaceutical extended release composition of claim 1, wherein the low molecular weight PEG is selected from the group consisting of PEG 250, PEG 300, PEG350, PEG 400, PEG 600, PEG 1000, PEG 1450, PEG 3350, and combinations thereof.

25. The pharmaceutical extended release composition of claim 1, wherein the low molecular weight PEG is PEG 300.

26. (canceled)

27. The pharmaceutical extended release composition of claim 1, wherein the biocompatible solvent is selected from the group consisting of N-methyl-2-pyrrolidone (NMP), dimethyl sulfoxide (DMSO), polyethylene glycol (PEG), butyrolactone, N-cycylohexyl-2-pyrrolidone, diethylene glycol monomethyl ether, dimethyl acetamide, dimethyl formamide, ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, polyoxyl 35 hydrogenated castor oil, polyoxyl 40 hydrogenated castor oil, benzyl alcohol, n-propanol, isopropanol, tert-butanol, propylene glycol, 2-pyrrolidone, triacetin, tributyl citrate, acetyl tributyl citrate, acetyl triethyl citrate, triethyl citrate, an ester of any of the foregoing, and combinations of any of the foregoing.

28. The pharmaceutical extended release composition of claim 1, wherein the biocompatible solvent is selected from the group consisting of from N-methyl-2-pyrrolidone (NMP), dimethyl sulfoxide (DMSO), and a combination thereof.

29. (canceled)

30. The pharmaceutical extended release composition of claim 1, wherein the solvent system comprises N-methyl-2-pyrrolidone and PEG 300.

31. (canceled)

32. (canceled)

33. (canceled)

34. The pharmaceutical extended release composition of claim 1, wherein the molar ratio of lactide to glycolide monomers is about 70:30-about 85:15.

35. The pharmaceutical extended release composition of claim 34, wherein the molar ratio of lactide to glycolide monomers is about 70:30.

36. (canceled)

37. (canceled)

38. (canceled)

39. The pharmaceutical extended release composition of claim 1, wherein the weight average molecular weight of the biodegradable polymer is about 4 kDa-about 14 kDa or is about 14 kDa-24 kDa.

40. (canceled)

41. (canceled)

42. The pharmaceutical extended release composition of claim 1, wherein the active pharmaceutical ingredient makes up from about 10 wt %-about 30 wt % of the pharmaceutical composition.

43. (canceled)

44. The pharmaceutical extended release composition of claim 1, wherein the solvent system makes up about 40 wt %-about 60 wt % of the pharmaceutical composition.

45. (canceled)

46. The pharmaceutical extended release composition of claim 1, wherein the biodegradable polymer makes up about 20 wt %-about 40 wt % of the pharmaceutical composition.

47. (canceled)

48. The pharmaceutical extended release composition claim 1, wherein the active pharmaceutical ingredient makes up about 20 wt % of the composition, the biocompatible solvent system makes up about 50 wt % of the composition, and the biodegradable polymer makes up about 30 wt % of the pharmaceutical composition.

49. (canceled)

50. (canceled)

51. (canceled)

52. (canceled)

53. A pharmaceutical extended release composition, comprising:
about 20 wt % of testosterone undecanoate having a D_{v,50}, prior to suspension in the pharmaceutical composition,of between about 35 μm-about 75 μm;
about 50 wt % of a biocompatible solvent system comprising N-methyl-2-pyrrolidone (NMP) and polyethylene glycol having a molecular weight of about 300 Daltons (PEG 300), wherein a weight ratio of NMP to PEG 300 is about 4:1; and
about 30 wt % of 70:30 poly(lactide-co-glycolide) (PLG) polymer having at least one carboxylic acid end group and having a weight average molecular weight of between about 4 kDa-about 14 kDa.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A method of testosterone replacement therapy for a condition associated with a deficiency or absence of endogenous testosterone in a subject, comprising administering to the subject the pharmaceutical extended release composition of claim 1.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. The method of claim 66, wherein upon administering the pharmaceutical composition to a subject, an average serum testosterone concentration of the subject is about 3 ng/mL-about 10 ng/mL for at least about one month after administration, about two months after administration, about three months after administration, about four months after administration or about five months after administration.

80. (canceled)

81. (canceled)

82. (canceled)

83. (canceled)

84. A syringe comprising the pharmaceutical composition of claim 1, wherein the syringe comprises an injection volume of about 2 mL or less.

85. (canceled)

86. (canceled)

87. (canceled)

88. (canceled)

89. (canceled)

90. (canceled)

91. The pharmaceutical extended release composition of claim 30, wherein a weight ratio of NMP to PEG 300 is about 4:1-to about 3:2.
